(19) United States
(12) Patent Application Publication
(10) Pub. No.: US 2019/0225680 A1
(43) Pub. Date: Jul. 25, 2019

(54) Tenascin Epitope and Antibodies Thereto

(71) Applicant: Nascient Ltd, London (GB)

(72) Inventors: Kim Midwood, Oxford (GB); Eric Culbert, Stevenage (GB); Patrick John Hextall, Cambridge (GB)

(73) Assignee: Nascient Ltd, London (GB)

(21) Appl. No.: 16/338,411

(22) PCT Filed: Sep. 29, 2017
(86) PCT No.: PCT/EP2017/074848

§ 371 Date: Mar. 29, 2019

(30) Foreign Application Priority Data
Sep. 29, 2016 (GB) 1616596.1

(51) Int. Cl. C07K 16/18

(52) U.S. Cl. CPC C07K 16/18; C07K 2317/76; C07K 2317/55; C07K 2317/33; C07K 2317/92; C07K 2317/34

(57) ABSTRACT

The present disclosure relates to a neutralising epitope for Tenascin-C, and binding domains specific thereto, for example antibodies or binding fragments thereof, pharmaceutical compositions comprising the same and use of said antibodies, binding fragments or composition in treatment, in particular the treatment of an inflammatory disorder.

The present disclosure relates to a neutralising epitope for Tenascin-C, and binding domains specific thereto, for example antibodies or binding fragments thereof, pharmaceutical compositions comprising the same and use of said antibodies, binding fragments or composition in treatment, in particular the treatment of an inflammatory disorder.

### BACKGROUND

Some work suggests that so-called loop 5, loop 7 and loop 10 of the P-subdomain of FBG domain of Tenascin-C are key epitopes for binding the TLR4 receptor. The interaction of the FBG domain of Tenascin-C with TLR4 is thought to be responsible for increasing the persistence of inflammatory responses in vivo. These areas of the FBG domain, in particular loop 5, are thought to be areas of interest for targeting with inhibitors, such as antibodies and binding fragments.

Surprisingly the present inventors have established that a conformational epitope, which does not include any residues from loop 5 neutralises the “inflammatory activity” of the FBG domain of Tenascin-C.

### SUMMARY OF THE DISCLOSURE

In one aspect there is provided a neutralising epitope of a Tenascin-C which comprises at least 5 amino acids independently selected from group Ala2215, Tyr2116, Asn2118, Ser2131, Ile2133, Tyr2140, Arg2147, Asn2148, Cys2149, His2150, Arg2151, His2163, Ser2164, Phe2170, His2171, and His2175, wherein said amino acid residue positions are defined re the protein disclosed in Uniprot ID: P24821 (SEQ ID NO: 1).

In one embodiment the epitope of the present disclosure is a conformational epitope.

In one embodiment the epitope is defined as amino acid residues located within 4 Å, 3.5 Å or 3.0 Å of a binding entity, such as an antibody or fragment, in particular the binding domain of antibody C3 disclosed herein.

In one embodiment the neutralising epitope of Tenascin-C according to the present disclosure comprises at least one charged residue Arg2147.

In one embodiment the neutralising epitope of Tenascin-C according to the present disclosure comprises a charged reside Arg2151.

In one embodiment the neutralising epitope of Tenascin-C according to the present disclosure comprises at least one hydrophobic residue Ile2133.

In one embodiment the neutralising epitope of Tenascin-C according to according to the comprises the linear sequence of amino acids Arg2147, Asn2148, Cys2149, His2150, and Arg2151.

In one embodiment the neutralising epitope of Tenascin-C according to the present disclosure comprises polar residue His2163.

In one embodiment the neutralising epitope of Tenascin-C according to the present disclosure comprises Cys2149.

In one embodiment the neutralising epitope of Tenascin-C according to comprises hydrophobic residue His2171.

In one embodiment the neutralising epitope of Tenascin-C according to the present disclosure comprises the polar amino acid Tyr2116.

In one embodiment the neutralising epitope of Tenascin-C according to the present disclosure comprises the hydrophobic residue Phe2170.

In one embodiment the neutralising epitope of Tenascin-C according to the present disclosure comprises Ile2133, Arg2147, Asn2148, His2163, and Phe2170.

In one embodiment the neutralising epitope of Tenascin-C according to the present disclosure further comprises amino acid residue Ser2131.

In one embodiment the neutralising epitope of Tenascin-C according to the present disclosure further comprises amino acid residue Tyr2216.

In one embodiment the neutralising epitope of Tenascin-C according to the present disclosure comprises 6, 7, 8, 9, 10, 11, 12, 13, 14 15 or 16 of said amino acid residues.

In one embodiment there is provided an inhibitor of the epitope, for example a small chemical entity, an antibody or binding fragment thereof.

In one embodiment there is provided a binding domain, for example in a neutralising antibody or binding fragment thereof, which binds an epitope according to the present disclosure.

The present invention also provides antibodies and binding fragments thereof which bind to, and/or interact with, a neutralising epitope provided by the present invention. It will be appreciated that an antibody can interact directly or indirectly with an epitope of the present invention, e.g. by direct binding or by allosteric interaction. In particular the antibodies and binding fragments, which form an aspect of the present disclosure have the one or more properties similar to the properties of the antibody C3, disclosed herein, such as affinity.

In one embodiment the binding domain according to the present disclosure is expressed on a cell surface, for example as part of a chimeric antigen receptor.

In one embodiment the neutralising antibody or binding fragment according to the present disclosure has at least one amino acid selected from residue 100, 101, 102, 104 and 105 of the antibody or binding fragment heavy chain which interacts with the epitope, in that it is located within 4 Ångströms of epitope, for example two, three, four or five of the residues interact with the epitope.

In one embodiment the amino acid residue 100 of the heavy chain of the antibody or binding fragment is polar, for example tyrosine.

In one embodiment amino acid 101 of the heavy chain of the antibody or binding fragment is a polar amino acid, for example Gln.

In one embodiment amino acid 102 of the heavy chain of the antibody or binding fragment is polar, for example serine.

In one embodiment amino acid 104 of the heavy chain of the antibody or binding fragment is charged, for example Glu.

In one embodiment amino acid 105 of the heavy chain of the antibody or binding fragment is a negatively charged amino acid, for example Asp.

In one embodiment the neutralising antibody or binding fragment according to the present disclosure has at least one amino acid selected from residue 28, 30, 31, 32, 50, 51, 52, 53, 64, 91 and 92 of the antibody or binding fragment light chain which interacts with the epitope, in that it is located within 4 Angstroms of epitope, for example two, three, four, five, six, seven, eight, nine, ten or eleven of the residues interact with the epitope.

In one embodiment the amino acid residue 28 of the light chain of the antibody or binding fragment is polar, for example tyrosine.

In one embodiment amino acid 30 of the light chain of the antibody or binding fragment is a polar amino acid, for example glutamine.

In one embodiment amino acid 31 of the light chain of the antibody or binding fragment is hydrophobic, for example glycine.

In one embodiment amino acid 32 of the light chain of the antibody or binding fragment is hydrophobic, for example phenylalanine.

In one embodiment amino acid 50 of the light chain of the antibody or binding fragment is a charged amino acid, for example aspartic acid.

In one embodiment amino acid 51 of the light chain of the antibody or binding fragment is hydrophobic, for example alanine.

In one embodiment amino acid 52 of the light chain of the antibody or binding fragment is hydrophobic, for example glycine.

In one embodiment amino acid 53 of the light chain of the antibody or binding fragment is a polar amino acid, for example asparagine.

In one embodiment amino acid 64 of the light chain of the antibody or binding fragment is hydrophobic, for example glycine.

In one embodiment amino acid 91 of the light chain of the antibody or binding fragment is polar, for example serine.

In one embodiment amino acid 92 of the light chain of the antibody or binding fragment is a polar amino acid, for example tyrosine.

In one embodiment the antibody or binding fragment is human or humanised.

In one embodiment the antibody or binding fragment has affinity is in the range 5 pM to 500 nM, for example 10, 20, 30, 40, 50, 60, 70, 80, 90, 100, 150, 200, 250, 300, 350, 400, 450, 500, 550, 600, 650, 700, 750, 800, 900 or 950 pM, 1, 2, 3, 4, 5, 6, 7, 8, 9, 10, 15, 20, 25, 30, 35, 40, 45, 50, 100, 150, 200, 250, 300, 350, 400 or 450 nM.

In one embodiment the antibody or binding fragment of the present disclosure does not comprise the CDRs of a C3 antibody.

In one embodiment the antibody or binding fragment of the present disclosure does not comprise the CDRs of a B12 antibody.

In one embodiment the antibody or binding fragment of the present disclosure does not comprise the CDRs of a 2A5 antibody.

Also provided is a pharmaceutical composition comprising an antibody or binding fragment according to the present disclosure and a diluent, excipient and/or carrier.

In one aspect the disclosure extends to a neutralising antibody or binding fragment or a pharmaceutical composition comprising any one of the same, for use in treatment, in particular for use in the treatment of chronic inflammation.

### DETAILED DISCLOSURE

Unless the context indicated otherwise the antibodies and binding fragments thereof comprise 6 CDR sequences.

“Does not comprise the CDRs of C3, B12 and 2A5” as employed herein refers to wherein at least one amino acid in any one of CDR H1, CDR H2, H3, L1, L2 L3 and combinations of two or more of the same, is substituted, added or deleted, such the CDR sequence is non-identical to a sequence disclosed herein (including the family members of said antibodies).

In one embodiment antibodies or binding fragments of the present disclosure comprise at least one CDR sequence which differs to the CDR sequences disclosed herein, in particular differs, for example by addition, substitution or deletion (in particular substitution or deletion) of at least one amino acid, such as 1, 2, 3, 4, 5, 6 or more amino acids in at least one CDR sequence.

In one embodiment CDR H1 is modified to different to a sequence disclosed herein.

In one embodiment CDR H2 is modified to different to a sequence disclosed herein.

In one embodiment CDR H3 is modified to different to a sequence disclosed herein.

In one embodiment CDR L1 is modified to different to a sequence disclosed herein.

In one embodiment CDR L2 is modified to different to a sequence disclosed herein.

In one embodiment CDR L3 is modified to different to a sequence disclosed herein.

In one embodiment two CDRs in the binding domain of the antibody or binding fragment differ, for example CDR H1 and H2; CDRH1 and H3; CDR H2 and H3; CDR L1 and L2; CDRL1 and L3; CDR L2 and L3; CDR H1 and CDR L1; CDR H2 and L1; CDR H3 and L1; CDR H1 and L2; CDR H2 and L2; CDR H3 and L2; CDR H1 and L3; CDR H2 and L3; CDR H3 and L3, differ from a sequence herein, for example by addition, substitution or deletion (in particular substitution or deletion) of at least one amino acid, such as 1, 2, 3, 4, 5, 6 or amino acids differ in at least one CDR sequence.

In one embodiment the three CDR in the binding domain of the antibody or binding fragment thereof differ, for example CDR H1, H2 and H3; CDR L1, L2 and L3, CDR H1, H2 and L1; CDR H1, H2 and L2; CDR H1, H2 and L3; CDR H1, H3 and L1; CDR H1, H3 and L2; CDR H1, H3 and L3; CDR H2, H3 and L1; CDR H2, H3 and L2; CDR H2, H3 and L3; CDR L1, L2 and H1; CDR L1, L2 and H2; CDR L1, L2 and H3; CDR L1, L3 and H1; CDR L1, L3 and H2; CDR L1, L3 and H3; CDR L2, L3 and H1; CDR L2, L3 and H2; CDR L2, L3 and H3, differ from a sequence herein, for example by addition, substitution or deletion (in particular substitution or deletion) of at least one amino acid, such as 1, 2, 3, 4, 5, 6 or amino acids differ in at least one CDR sequence.

In one embodiment 4 CDRs in the binding domain differ from the corresponding antibody CDRs disclosed herein.

In one embodiment 5 CDRs in the binding domain differ from the corresponding antibody CDRs disclosed herein.

In one embodiment 6 CDRs in the binding domain differs for the corresponding antibody CDRs disclosed herein.

In one embodiment at least one, such as 1, 2, 3, 4, 5 or 6 CDRs differ from CDR H1 is SEQ ID NO: 27, CDR H2 is SEQ ID NO: 28, CDR H3 is SEQ ID NO: 29. CDR L1 is SEQ ID NO: 7, CDR L2 is SEQ ID NO: 8, CDR L3 is SEQ ID NO: 31.

In one embodiment at least one, such as 1, 2, 3, 4, 5 or 6 CDRs differ from CDR H1 is SEQ ID NO: 34, CDR H2 is SEQ ID NO: 35, CDR H3 is SEQ ID NO: 36. CDR L1 is SEQ ID NO: 38, CDR L2 is SEQ ID NO: 39, CDR L3 is SEQ ID NO: 40.

In one embodiment at least one, such as 1, 2, 3, 4, 5 or 6 CDRs differ from CDR H1 is SEQ ID NO: 4, CDR H2 is SEQ ID NO: 5, CDR H3 is SEQ ID NO: 22. CDR L1 is SEQ ID NO: 7, CDR L2 is SEQ ID NO: 8, CDR L3 is SEQ ID NO: 24.

In one embodiment at least one, such as 1, 2, 3, 4, 5 or 6 CDRs differ from CDR H1 is SEQ ID NO: 4, CDR H2 is SEQ ID NO: 5, CDR H3 is SEQ ID NO: 43. CDR L1 is SEQ ID NO: 7, CDR L2 is SEQ ID NO: 8, CDR L3 is SEQ ID NO: 9.

In one embodiment at least one, such as 1, 2, 3, 4, 5 or 6 CDRs differ from CDR H1 is SEQ ID NO: 4, CDR H2 is SEQ ID NO: 5, CDR H3 is SEQ ID NO: 46. CDR L1 is SEQ ID NO: 7, CDR L2 is SEQ ID NO: 8, CDR L3 is SEQ ID NO: 9.

In one embodiment at least one, such as 1, 2, 3, 4, 5 or 6 CDRs differ from CDR H1 is SEQ ID NO: 4, CDR H2 is SEQ ID NO: 5, CDR H3 is SEQ ID NO: 49. CDR L1 is SEQ ID NO: 7, CDR L2 is SEQ ID NO: 8, CDR L3 is SEQ ID NO: 9.

In one embodiment at least one, such as 1, 2, 3, 4, 5 or 6 CDRs differ from CDR H1 is SEQ ID NO: 4, CDR H2 is SEQ ID NO: 5, CDR H3 is SEQ ID NO: 6. CDR L1 is SEQ ID NO: 7, CDR L2 is SEQ ID NO: 8, CDR L3 is SEQ ID NO: 9.

In one embodiment at least one, such as 1, 2, 3, 4, 5 or 6 CDRs differ from CDR H1 is SEQ ID NO: 4, CDR H2 is SEQ ID NO: 5, CDR H3 is SEQ ID NO: 53. CDR L1 is SEQ ID NO: 7, CDR L2 is SEQ ID NO: 8, CDR L3 is SEQ ID NO: 9.

In one embodiment at least one, such as 1, 2, 3, 4, 5 or 6 CDRs differ from CDR H1 is SEQ ID NO: 4, CDR H2 is SEQ ID NO: 5, CDR H3 is SEQ ID NO: 15. CDR L1 is SEQ ID NO: 7, CDR L2 is SEQ ID NO: 8, CDR L3 is SEQ ID NO: 9.

In one embodiment at least one, such as 1, 2, 3, 4, 5 or 6 CDRs differ from CDR H1 is SEQ ID NO: 4, CDR H2 is SEQ ID NO: 5, CDR H3 is SEQ ID NO: 21. CDR L1 is SEQ ID NO: 7, CDR L2 is SEQ ID NO: 8, CDR L3 is SEQ ID NO: 9.

In one embodiment at least one, such as 1, 2, 3, 4, 5 or 6 CDRs differ from CDR H1 is SEQ ID NO: 4, CDR H2 is SEQ ID NO: 5, CDR H3 is SEQ ID NO: 45. CDR L1 is SEQ ID NO: 7, CDR L2 is SEQ ID NO: 8, CDR L3 is SEQ ID NO: 9.

In one embodiment at least one, such as 1, 2, 3, 4, 5 or 6 CDRs differ from CDR H1 is SEQ ID NO: 4, CDR H2 is SEQ ID NO: 5, CDR H3 is SEQ ID NO: 51. CDR L1 is SEQ ID NO: 7, CDR L2 is SEQ ID NO: 8, CDR L3 is SEQ ID NO: 9.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 56. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 58. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 60. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 62. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 64. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 66. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 68. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 70. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 72. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 74. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 76. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 78. CDR L1 is SEQ ID NO: 7, CDR L2 is SEQ ID NO: 16, CDR L3 is SEQ ID NO: 17.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 80.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 83.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 86.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 89.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 92.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 95.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 98.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 101 or 102.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 105.

In one embodiment at least one, such as 1, 2, 3, 4, 5 or 6 CDRs differ from CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 108.

Differs as employed herein refers to addition, substitution or deletion (in particular substitution or deletion) of at least one amino acid, such as 1, 2, 3, 4, 5, 6 or more amino acids in at least one CDR sequence, such as 1, 2, 3, 4, 5 or 6 CDRs.

“Neutralising epitope’ as employed herein refers to an epitope that can be inhibited or blocked thereby neutralising the biological signalling activity of the FBG domain of a Tenascin (such as Tenascin-C), in particular the P-subdomain thereof, to one or more of its receptors, for example TLR4.

As used herein, the term ‘neutralising antibody’ describes an antibody that is capable of neutralising the biological signalling activity of the FBG domain of a Tenascin (such as Tenascin-C), in particular the P-subdomain thereof, to one or more of its receptors, for example TLR4.

It will be appreciated that the term ‘neutralising’ as used herein refers to a reduction in biological signalling activity which may be partial or complete.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al. (supra)”). This numbering system is used in the present specification except where otherwise indicated.

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.

The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus ‘CDR-H1’, as used herein, comprises residues 26 to 35, as described by a combination of the Kabat numbering system and Chothia's topological loop definition.

The CDRs of the light chain variable domain are located at residues 24-34 (CDR-L1), residues 50-56 (CDR-L2) and residues 89-97 (CDR-L3) according to the Kabat numbering system.

Antibodies for use in the present invention include whole antibodies and functionally active fragments or derivatives thereof and may be, but are not limited to, monoclonal, multi-valent, multi-specific, fully human, humanized or chimeric antibodies, domain antibodies e.g. VH, VL, VHH, single chain antibodies, Fab fragments, Fab′ and F(ab′)₂ fragments and epitope-binding fragments of any of the above. Other antibody fragments include those described in International patent applications WO2005003169, WO2005003170 and WO2005003171. Antibody fragments and methods of producing them are well known in the art, see for example Verma et al., 1998, Journal of Immunological Methods, 216, 165-181; Adair and Lawson, 2005. Therapeutic antibodies. Drug Design Reviews—Online 2(3):209-217.

Antibodies for use in the present invention include immunoglobulin molecules and immunologically active portions of immunoglobulin molecules, i.e. molecules that contain an antigen binding site that specifically binds an antigen. The immunoglobulin molecules of the invention can be of any class (e.g. IgG, IgE, IgM, IgD and IgA) or subclass of immunoglobulin molecule. The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required, e.g. for simply blocking activity. It will be appreciated that sequence variants of these constant region domains may also be used. For example IgG4 molecules in which the serine at position 241 has been changed to proline as described in Angal et al., Molecular Immunology, 1993, 30 (1), 105-108 may be used. Particularly preferred is the IgG4 constant domain comprising this change. It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, R J. Journal of Chromatography 705:129-134, 1995).

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp77-96, Alan R Liss, Inc., 1985).

Antibodies for use in the invention may also be generated using single lymphocyte antibody methods by cloning and expressing immunoglobulin variable region cDNAs generated from single lymphocytes selected for the production of specific antibodies by for example the methods described by Babcook, J. et al., 1996, Proc. Natl. Acad. Sci. USA 93(15):7843-78481; WO92/02551; WO2004/051268 and International Patent Application number WO2004/106377.

Humanized antibodies are antibody molecules from non-human species having one or more complementarity determining regions (CDRs) from the non-human species and a framework region from a human immunoglobulin molecule (see, e.g. U.S. Pat. No. 5,585,089; WO91/09967).

Chimeric antibodies are those antibodies encoded by immunoglobulin genes that have been genetically engineered so that the light and heavy chain genes are composed of immunoglobulin gene segments belonging to different species. These chimeric antibodies are likely to be less antigenic.

Bivalent antibodies may be made by methods known in the art (Milstein et al., 1983, Nature 305:537-539; WO 93/08829, Traunecker et al., 1991, EMBO J. 10:3655-3659). Multi-valent antibodies may comprise multiple specificities or may be monospecific (see for example WO 92/22853 and WO05/113605).

The antibodies for use in the present invention can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. Techniques for the production of single chain antibodies, such as those described in U.S. Pat. No. 4,946,778 can also be adapted to produce single chain antibodies which bind the epitope according to the present disclosure. Also, transgenic mice, or other organisms, including other mammals, may be used to express humanized antibodies.

Antibodies and binding fragments disclosed herein can be mutated to and engineered and/or affinity matured and/or light chains swapped to provide new antibodies and binding fragments. Alternatively, idiopathic antibodies can be generated specific to a binding site of an antibody disclosed herein. These idiopathic antibodies generated in this way may have an increased propensity to bind the same epitope as the original antibody of the present disclosure.

Fully human antibodies are those antibodies in which the variable regions and the constant regions (where present) of both the heavy and the light chains are all of human origin, or substantially identical to sequences of human origin, not necessarily from the same antibody. Examples of fully human antibodies may include antibodies produced for example by the phage display methods described above and antibodies produced by mice in which the murine immunoglobulin variable and constant region genes have been replaced by their human counterparts eg. as described in general terms in EP0546073 B1, U.S. Pat. Nos. 5,545,806, 5,569,825, 5,625,126, 5,633,425, 5,661,016, 5,770,429, EP 0438474 B1 and EP0463151B1.

Any suitable method known in the art may be used to determine the residues bound by an antibody provided by the present invention e.g. hydrogen-deuterium exchange, site-directed mutagenesis, mass spectrometry, NMR and X-ray crystallography. See for example the methods described in WO2007/149032.

If desired an antibody for use in the present invention may be conjugated to one or more effector molecule (s). It will be appreciated that the effector molecule may comprise a single effector molecule or two or more such molecules, for example linked to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO03031581. Alternatively, where the effector molecule is a protein or polypeptide the linkage may be achieved using recombinant DNA procedures, for example as described in WO 86/01533 and EP0392745.

The term effector molecule as used herein includes, for example, antineoplastic agents, drugs, toxins, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Examples of effector molecules may include cytotoxins or cytotoxic agents including any agent that is detrimental to (e.g. kills) cells. Examples include combrestatins, dolastatins, epothilones, staurosporin, maytansinoids, spongistatins, rhizoxin, halichondrins, roridins, hemiasterlins, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Effector molecules also include, but are not limited to, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine). Other effector molecules may include chelated radionuclides such as ¹¹¹In and ⁹⁰Y, Lu¹⁷⁷, Bismuth²¹³, Californium²⁵², Iridium¹⁹² and Tungsten¹⁸⁸/Rhenium¹⁸⁸; or drugs such as but not limited to, alkylphosphocholines, topoisomerase I inhibitors, taxoids and suramin.

Other effector molecules include proteins, peptides and enzymes. Enzymes of interest include, but are not limited to, proteolytic enzymes, hydrolases, lyases, isomerases, transferases. Proteins, polypeptides and peptides of interest include, but are not limited to, immunoglobulins, toxins such as abrin, ricin A, pseudomonas exotoxin, or diphtheria toxin, a protein such as insulin, tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor and immunoglobulins.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase;

suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include ¹²⁵I, ¹³¹I, ¹¹¹In and ⁹⁹Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984.

Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Particular optional substituents which may be present on the above-mentioned synthetic polymers include one or more hydroxy, methyl or methoxy groups.

Particular examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Particular naturally occurring polymers include lactose, amylose, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired, but will generally be in an average molecular weight range from 500 Da to 50000 Da, preferably from 5000 to 40000 Da and more preferably from 20000 to 40000 Da. The polymer size may in particular be selected on the basis of the intended use of the product for example ability to localize to certain tissues such as tumors or extend circulating half-life (for review see Chapman, 2002, Advanced Drug Delivery Reviews, 54, 531-545). Thus, for example, where the product is intended to leave the circulation and penetrate tissue, for example for use in the treatment of a tumour, it may be advantageous to use a small molecular weight polymer, for example with a molecular weight of around 5000 Da. For applications where the product remains in the circulation, it may be advantageous to use a higher molecular weight polymer, for example having a molecular weight in the range from 20000 Da to 40000 Da.

Particularly preferred polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971).

In one example the antibody molecule of the present invention is a modified Fab fragment wherein the modification is the addition to the C-terminal end of its heavy chain one or more amino acids to allow the attachment of an effector molecule. Preferably, the additional amino acids form a modified hinge region containing one or more cysteine residues to which the effector molecule may be attached. Multiple sites can be used to attach two or more PEG molecules.

Preferably PEG molecules are covalently linked through a thiol group of at least one cysteine residue located in the antibody fragment. Each polymer molecule attached to the modified antibody fragment may be covalently linked to the sulphur atom of a cysteine residue located in the fragment. The covalent linkage will generally be a disulphide bond or, in particular, a sulphur-carbon bond. Where a thiol group is used as the point of attachment appropriately activated effector molecules, for example thiol selective derivatives such as maleimides and cysteine derivatives may be used. An activated polymer may be used as the starting material in the preparation of polymer-modified antibody fragments as described above. The activated polymer may be any polymer containing a thiol reactive group such as an α-halocarboxylic acid or ester, e.g. iodoacetamide, an imide, e.g. maleimide, a vinyl sulphone or a disulphide. Such starting materials may be obtained commercially (for example from Nektar, formerly Shearwater Polymers Inc., Huntsville, Ala., USA) or may be prepared from commercially available starting materials using conventional chemical procedures. Particular PEG molecules include 20K methoxy-PEG-amine (obtainable from Nektar, formerly Shearwater; Rapp Polymere; and SunBio) and M-PEG-SPA (obtainable from Nektar, formerly Shearwater).

In one embodiment, the antibody is a modified Fab fragment which is PEGylated, i.e. has PEG (poly(ethyleneglycol)) covalently attached thereto, e.g. according to the method disclosed in EP 0948544 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington D.C. and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one example PEG is attached to a cysteine in the hinge region. In one example, a PEG modified Fab fragment has a maleimide group covalently linked to a single thiol group in a modified hinge region. A lysine residue may be covalently linked to the maleimide group and to each of the amine groups on the lysine residue may be attached a methoxypoly(ethyleneglycol) polymer having a molecular weight of approximately 20,000 Da. The total molecular weight of the PEG attached to the Fab fragment may therefore be approximately 40,000 Da.

In one embodiment, a neutralising antibody molecule of the present invention is a modified Fab fragment having at the C-terminal end of its heavy chain a modified hinge region containing at least one cysteine residue to which an effector molecule is attached. Preferably the effector molecule is PEG and is attached using the methods described in (WO98/25971 and WO2004072116) whereby a lysyl-maleimide group is attached to the cysteine residue at the C-terminal end of the heavy chain, and each amino group of the lysyl residue has covalently linked to it a methoxypoly(ethyleneglycol) residue having a molecular weight of about 20,000 Da. The total molecular weight of the PEG attached to the antibody is therefore approximately 40,000 Da.

In another example effector molecules may be attached to antibody fragments using the methods described in International patent applications WO2005/003169, WO2005/003170 and WO2005/003171.

The present invention also provides an isolated DNA sequence encoding the heavy and/or light chain(s) of an antibody molecule of the present invention. Preferably, the DNA sequence encodes the heavy or the light chain of an antibody molecule of the present invention. The DNA sequence of the present invention may comprise synthetic DNA, for instance produced by chemical processing, cDNA, genomic DNA or any combination thereof.

DNA sequences which encode an antibody molecule of the present invention can be obtained by methods well known to those skilled in the art. For example, DNA sequences coding for part or all of the antibody heavy and light chains may be synthesised as desired from the determined DNA sequences or on the basis of the corresponding amino acid sequences.

DNA coding for acceptor framework sequences is widely available to those skilled in the art and can be readily synthesised on the basis of their known amino acid sequences.

Standard techniques of molecular biology may be used to prepare DNA sequences coding for the antibody molecule of the present invention. Desired DNA sequences may be synthesised completely or in part using oligonucleotide synthesis techniques. Site-directed mutagenesis and polymerase chain reaction (PCR) techniques may be used as appropriate.

The present invention also relates to a cloning or expression vector comprising one or more DNA sequences of the present invention. Accordingly, provided is a cloning or expression vector comprising one or more DNA sequences encoding an antibody of the present invention. Preferably, the cloning or expression vector comprises two DNA sequences, encoding the light chain and the heavy chain of the antibody molecule of the present invention, respectively.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

Also provided is a host cell comprising one or more cloning or expression vectors comprising one or more DNA sequences encoding an antibody of the present invention. Any suitable host cell/vector system may be used for expression of the DNA sequences encoding the antibody molecule of the present invention. Bacterial, for example E. coli, and other microbial systems may be used or eukaryotic, for example mammalian, host cell expression systems may also be used. Suitable mammalian host cells include CHO, myeloma or hybridoma cells.

The present invention also provides a process for the production of an antibody molecule according to the present invention comprising culturing a host cell containing a vector of the present invention under conditions suitable for leading to expression of protein from DNA encoding the antibody molecule of the present invention, and isolating the antibody molecule.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

As the antibodies of the present invention are useful in the treatment and/or prophylaxis of a pathological condition, the present invention also provides a pharmaceutical or diagnostic composition comprising an antibody molecule of the present invention in combination with one or more of a pharmaceutically acceptable excipient, diluent or carrier. Accordingly, provided is the use of an antibody according to the present invention for the manufacture of a medicament. The composition will usually be supplied as part of a sterile, pharmaceutical composition that will normally include a pharmaceutically acceptable carrier. A pharmaceutical composition of the present invention may additionally comprise a pharmaceutically-acceptable adjuvant.

The present invention also provides a process for preparation of a pharmaceutical or diagnostic composition comprising adding and mixing the antibody molecule of the present invention together with one or more of a pharmaceutically acceptable excipient, diluent or carrier.

The antibody molecule may be the sole active ingredient in the pharmaceutical or diagnostic composition or may be accompanied by other active ingredients including other antibody ingredients, for example anti-TNF, anti-IL-1β, anti-T cell, anti-IFNγ or anti-LPS antibodies, or non-antibody ingredients such as xanthines.

The pharmaceutical compositions preferably comprise a therapeutically effective amount of the antibody of the invention. The term “therapeutically effective amount” as used herein refers to an amount of a therapeutic agent needed to treat, ameliorate or prevent a targeted disease or condition, or to exhibit a detectable therapeutic or preventative effect. For any antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

The precise therapeutically effective amount for a human subject will depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount will be from 0.01 mg/kg to 50 mg/kg, preferably 0.1 mg/kg to 20 mg/kg. Pharmaceutical compositions may be conveniently presented in unit dose forms containing a predetermined amount of an active agent of the invention per dose.

Compositions may be administered individually to a patient or may be administered in combination (e.g. simultaneously, sequentially or separately) with other agents, drugs or hormones.

The dose at which the antibody molecule of the present invention is administered depends on the nature of the condition to be treated, the extent of the inflammation present and on whether the antibody molecule is being used prophylactically or to treat an existing condition.

The frequency of dose will depend on the half-life of the antibody molecule and the duration of its effect. If the antibody molecule has a short half-life (e.g. 2 to 10 hours) it may be necessary to give one or more doses per day. Alternatively, if the antibody molecule has a long half life (e.g. 2 to 15 days) it may only be necessary to give a dosage once per day, once per week or even once every 1 or 2 months.

The pharmaceutically acceptable carrier should not itself induce the production of antibodies harmful to the individual receiving the composition and should not be toxic. Suitable carriers may be large, slowly metabolised macromolecules such as proteins, polypeptides, liposomes, polysaccharides, polylactic acids, polyglycolic acids, polymeric amino acids, amino acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the patient.

Preferred forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the antibody molecule may be in dry form, for reconstitution before use with an appropriate sterile liquid, for example water for injection, saline, glucose or similar.

Once formulated, the compositions of the invention can be administered directly to the subject. The subjects to be treated may be animals. However, it is preferred that the compositions are adapted for administration to human subjects. Thus in one embodiment the patient is a human.

The pharmaceutical compositions of this invention may be administered by any number of routes including, but not limited to, oral, intravenous, intramuscular, intra-arterial, intramedullary, intrathecal, intraventricular, transdermal, transcutaneous (for example, see WO 98/20734), subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion.

Dosage treatment may be a single dose schedule or a multiple dose schedule.

It will be appreciated that the active ingredient in the composition will be an antibody molecule. As such, it will be susceptible to degradation in the gastrointestinal tract. Thus, if the composition is to be administered by a route using the gastrointestinal tract, the composition will need to contain agents which protect the antibody from degradation but which release the antibody once it has been absorbed from the gastrointestinal tract.

A thorough discussion of pharmaceutically acceptable carriers is available in Remington's Pharmaceutical Sciences (Mack Publishing Company, N.J. 1991).

It is also envisaged that the antibody of the present invention will be administered by use of gene therapy. In order to achieve this, DNA sequences encoding the heavy and light chains of the antibody molecule under the control of appropriate DNA components are introduced into a patient such that the antibody chains are expressed from the DNA sequences and assembled in situ.

The present invention also provides an antibody or binding fragment thereof for use in the control of inflammatory diseases. Preferably, the antibody molecule can be used to reduce the inflammatory process or to prevent the inflammatory process.

Inflammatory process includes a chronic inflammatory condition wherein the condition is associated with any condition associated with inappropriate inflammation. Such conditions include, but are not limited to, rheumatoid arthritis (RA), autoimmune conditions, inflammatory bowel diseases (including Crohn's disease and ulcerative colitis), non-healing wounds, multiple sclerosis, cancer, atherosclerosis, sjogrens disease, diabetes, lupus erythrematosus (including systemic lupus erythrematosus), asthma, fibrotic diseases (including liver cirrhosis), pulmonary fibrosis, UV damage, psoriasis, ankylosing spondylitis, cardiovascular disease, alzheimer's disease and parkinson's disease.

In the context of this specification “comprising” is to be interpreted as “including”.

Embodiments of the invention comprising certain features/elements are also intended to extend to alternative embodiments “consisting” or “consisting essentially” of the relevant elements/features.

Where technically appropriate, embodiments of the invention may be combined. Technical references such as patents and applications are incorporated herein by reference.

Any embodiments specifically and explicitly recited herein may form the basis of a disclaimer either alone or in combination with one or more further embodiments.

The present invention is further described by way of illustration only in the following examples.

### DESCRIPTION OF FIGURES

FIG. 1 is a chromatograph showing the formation of the Fab′-Tenascin complex at an optimised molar ratio of 1:1

FIG. 2A is a chromatograph showing the successful formation of the Fab′-Tenascin complex.

FIGS. 2B and C are photos of western blots showing the results of SDS-PAGE analysis to confirm that the Fab′-Tenascin complex is of the correct size.

FIG. 3 shows images of a crystal of the Fab′-Tenascin complex A) taken under white light B) taken under UV light.

FIG. 4 is an image of a single well-diffracting crystal of the Fab′-Tenascin complex produced using the final optimised process conditions.

FIG. 5 is an image of a computer model of the Fab′-Tenascin complex.

FIG. 6 is an image showing the Fab′-Tenascin complex and the residues of interest on the Tenascin C complex. A) overview showing all residues of interest. B) residues 2096-2102 C) residues 2130-2135 D) residues 2170-2193

FIG. 7 shows a computer model of the Fab′-Tenascin complex. A) surface representation of complex B) residues of interest on Tenascin C) and D) close up of point of interaction between Fab′ and tenascin C.

FIG. 8 shows a computer model of the Fab′-Tenascin complex. The residues of interest (within 4 Å of the tenascin construct) on the Fab′ are shown.

### SEQUENCES

SEQ ID NO: 1 Tenascin C amino acid sequence Uniprot ID No: P24821
SEQ ID NO: 2 Fab′ heavy chain amino acid sequence of the C3 antibody
SEQ ID NO: 3 Fab′ light chain amino acid sequence of the C3 antibody
SEQ ID NO: 4 Fab′ VH of SEQ ID NO: 2 CDR1 amino acid sequence (this sequence is also CDR H1 in antibody B12)
SEQ ID NO: 5 Fab′ VH of SEQ ID NO: 2 CDR2 amino acid sequence (this sequence is also CDR H2 in antibody B12)
SEQ ID NO: 6 Fab′ VH of SEQ ID NO: 2 CDR3 amino acid sequence
SEQ ID NO: 7 Fab′ VL of SEQ ID NO: 3 CDR1 amino acid sequence (this sequence is also CDR L1 of antibody 2A5, antibody B12, antibody D8)
SEQ ID NO: 8 Fab′ VL of SEQ ID NO: 3 CDR2 amino acid sequence (this sequence is also the CDR L2 of antibody B12 and antibody D8)
SEQ ID NO: 9 Fab′ VL of SEQ ID NO: 3 CDR3 amino acid sequence
SEQ ID NO: 10 Tenascin C construct amino acid sequence (also referred to herein as a TNC fragment)
SEQ ID NO: 11 Is CDR H1 from antibody 2A5
SEQ ID NO: 12 Is CDR H2 from antibody 2A5
SEQ ID NO: 13 Is CDR H3 from antibody 2A5
SEQ ID NO: 14 Is the VH domain of antibody 2A5
SEQ ID NO: 15 Is CDR H3 of antibody A4
SEQ ID NO: 16 Is CDR L2 from antibody 2A5
SEQ ID NO: 17 Is CDR L3 from antibody 2A5
SEQ ID NO: 18 Is the VL domain of antibody 2A5
SEQ ID NO: 19 Is an alternative VL domain for antibody 2A5
SEQ ID NO: 20 Is the VH domain of antibody A4
SEQ ID NO: 21 Is CDR H3 of antibody B3
SEQ ID NO: 22 Is CDR L3 of antibody B12.
SEQ ID NO: 23 Is a VH domain of antibody B12
SEQ ID NO: 24 Is the VH domain of antibody B3
SEQ ID NO: 25 Is the VL domain of antibody B12
SEQ ID NO: 26 Is an alternative VL domain of antibody B12
SEQ ID NO: 27 Is CDR H1 of antibody D8
SEQ ID NO: 28 Is CDR H2 of antibody D8
SEQ ID NO: 29 Is CDR H3 of antibody D8
SEQ ID NO: 30 Is the VH domain of antibody D8
SEQ ID NO: 31 Is the CDR L3 for antibody D8
SEQ ID NO: 32 Is the VL domain for antibody D8
SEQ ID NO: 33 Is an alternative VL domain for antibody D8
SEQ ID NO: 34 Is CDR H1 for antibody F3
SEQ ID NO: 35 Is CDR H2 for antibody F3
SEQ ID NO: 36 Is CDR H3 for antibody F3
SEQ ID NO: 37 Is the VH domain for antibody F3
SEQ ID NO: 38 Is CDR L1 of antibody F3
SEQ ID NO: 39 Is CDR L2 of antibody F3
SEQ ID NO: 40 Is CDR L3 of antibody F3
SEQ ID NO: 41 Is the VL domain of antibody F3
SEQ ID NO: 42 Is an alternative VL domain of antibody F3
SEQ ID NO: 43 Is CDR H3 for antibody B1
SEQ ID NO: 44 Is the VH domain of antibody B1
SEQ ID NO: 45 Is CDR H3 of antibody E1
SEQ ID NO: 46 Is CDR H3 of antibody B6
SEQ ID NO: 47 Is the VH domain of antibody B6
SEQ ID NO: 48 Is the VH domain of antibody E1
SEQ ID NO: 49 Is CDR H3 of antibody D1
SEQ ID NO: 50 Is the VH domain of antibody D1
SEQ ID NO: 51 Is CDR H3 of antibody F5
SEQ ID NO: 52 Is the VH domain of antibody C3
SEQ ID NO: 53 Is CDR H3 of antibody D4
SEQ ID NO: 54 Is the VH domain of antibody D4
SEQ ID NO: 55 Is the VH domain of antibody F5
SEQ ID NO: 56 Is CDR H3 of antibody E3
SEQ ID NO: 57 Is the VH domain of antibody E3
SEQ ID NO: 58 Is CDR H3 of antibody D6
SEQ ID NO: 59 Is the VH domain of antibody D6
SEQ ID NO: 60 Is CDR H3 of antibody H4
SEQ ID NO: 61 Is the VH domain of antibody H4
SEQ ID NO: 62 Is CDR H3 of antibody A4
SEQ ID NO: 63 Is the VH domain of antibody A4
SEQ ID NO: 64 Is CDR H3 of antibody F1
SEQ ID NO: 65 Is the VH domain of antibody F1
SEQ ID NO: 66 Is CDR H3 of antibody G2
SEQ ID NO: 67 Is the VH domain of antibody G2
SEQ ID NO: 68 Is CDR H3 of antibody F6
SEQ ID NO: 69 Is the VH domain of F6
SEQ ID NO: 70 Is CDR H3 of antibody A12
SEQ ID NO: 71 Is the VH domain of antibody A12
SEQ ID NO: 72 Is CDR H3 of antibody C09
SEQ ID NO: 73 Is the VH domain of antibody C09
SEQ ID NO: 74 Is CDR H3 of antibody H10
SEQ ID NO: 75 Is the VH domain of antibody H10
SEQ ID NO: 76 Is CDR H3 of antibody C11
SEQ ID NO: 77 Is the VH domain of antibody C11
SEQ ID NO: 78 Is CDR H3 of antibody D3
SEQ ID NO: 79 Is the VH domain of antibody D3
SEQ ID NO: 80 Is CDR L3 of antibody C6
SEQ ID NO: 81 Is the VL domain of antibody C6
SEQ ID NO: 82 Is an alternative VL domain of antibody C6
SEQ ID NO: 83 Is CDR L3 of antibody H5
SEQ ID NO: 84 Is the VL domain of antibody H5
SEQ ID NO: 85 Is an alternative VL domain of antibody H5
SEQ ID NO: 86 Is CDR L3 of antibody F3
SEQ ID NO: 87 Is the VL domain of antibody F3
SEQ ID NO: 88 Is an alternative VL domain of antibody F3
SEQ ID NO: 89 Is CDR L3 of antibody C1
SEQ ID NO: 90 Is the VL domain of antibody C1
SEQ ID NO: 91 Is an alternative VL domain of antibody C1
SEQ ID NO: 92 Is CDR L3 of antibody C2
SEQ ID NO: 93 Is the VL domain of antibody C2
SEQ ID NO: 94 Is an alternative VL domain of antibody C2
SEQ ID NO: 95 Is CDR L3 of antibody F4
SEQ ID NO: 96 Is the VL domain of antibody F4
SEQ ID NO: 97 Is an alternative VL domain of antibody F4
SEQ ID NO: 98 Is CDR L3 of antibody C3
SEQ ID NO: 99 Is the VL domain of antibody C3
SEQ ID NO: 100 Is an alternative VL domain of antibody C3
SEQ ID NO: 101 Is CDR L3 of antibody E11
SEQ ID NO: 102 Is an alternative CDR L3 of antibody E11
SEQ ID NO: 103 Is the VL domain of antibody E11
SEQ ID NO: 104 Is an alternative VL domain of antibody E11
SEQ ID NO: 105 Is an alternative CDR L3 of antibody A12
SEQ ID NO: 106 Is the VL domain of antibody A12
SEQ ID NO: 107 Is an alternative VL domain of antibody A12
SEQ ID NO: 108 Is an alternative CDR L3 of antibody D11
SEQ ID NO: 109 Is the VL domain of antibody D11
SEQ ID NO: 110 Is an alternative VL domain of antibody D11
SEQ ID NO: 111 Is an IgG4 format of antibody C3 with hinge modification as described in Angal 1993
SEQ ID NO: 112 Human tenascin-C FBG domain
SEQ ID NO: 113 Murine tenascin-C FBG domain
SEQ ID NO: 114 Rat tenascin-C FBG domain
SEQ ID NO: 115 Canine tenascin-C FBG domain
SEQ ID NO: 116 Human tenascin-R FBG domain
SEQ ID NO: 117 Amino acid sequence of germlined VH framework (Antibody 2A5)
SEQ ID NO: 118 Amino acid sequence of germlined VL framework (Antibody 2A5)
SEQ ID NO: 119 Amino acid sequence of germlined VL framework (Antibody 2A5)
SEQ ID NO: 120 Alternative CDR L2 for 2A5 and 2A5 family of antibodies
SEQ ID NO: 121 Sequence of germlined VH framework (Antibody B12)
SEQ ID NO: 122 Alternative CDR H2 for B12 and B12 family of antibodies
SEQ ID NO: 123 Amino acid sequence of germlined VL framework (Antibody B12)
SEQ ID NO: 124 Amino acid sequence of germlined VL framework (Antibody B12)
SEQ ID NO: 125 Alternative CDR L2 for B12, D8 and B12 family of antibodies
SEQ ID NO: 126 Amino acid sequence of germlined VH framework (Antibody D8)
SEQ ID NO: 127 Amino acid sequence of germlined VL framework (Antibody D8)
SEQ ID NO: 128 Amino acid sequence of germlined VL framework (Antibody D8)
SEQ ID NO: 129 Amino acid sequence of germlined VH framework (Antibody F3)
SEQ ID NO: 130 Amino acid sequence of germlined VL framework (Antibody F3)
SEQ ID NO: 131 Amino acid sequence of germlined VL framework (Antibody F3)
SEQ ID NO: 132-134 Fragments of Tenascin-C
SEQ ID NO: 135-148 Primer sequences
   Closest germline matches were determined using IMGT/DomainGapAlign:

### Ehrenmann F., Kaas Q. and Lefranc M. P. Nucleic Acids Res., 38, D301-307 (2010)

Antibody D8: CDR H1 is SEQ ID NO: 27, CDR H2 is SEQ ID NO: 28, CDR H3 is SEQ ID NO: 29. CDR L1 is SEQ ID NO: 7, CDR L2 is SEQ ID NO: 8, CDR L3 is SEQ ID NO: 31. The VH is SEQ ID NO: 30 and the VL is SEQ ID NO: 32 or 33.
Antibody F3′: CDR H1 is SEQ ID NO: 34, CDR H2 is SEQ ID NO: 35, CDR H3 is SEQ ID NO: 36. CDR L1 is SEQ ID NO: 38, CDR L2 is SEQ ID NO: 39, CDR L3 is SEQ ID NO: 40. The VH is SEQ ID NO: 37 and the VL is SEQ ID NO: 41 or 42.

### B12 Antibody Family

Antibody B12 CDR H1 is SEQ ID NO: 4, CDR H2 is SEQ ID NO: 5, CDR H3 is SEQ ID NO: 22. CDR L1 is SEQ ID NO: 7, CDR L2 is SEQ ID NO: 8, CDR L3 is SEQ ID NO: 24. The VH is SEQ ID NO: 23 and the VL is SEQ ID NO: 25 or 26.
Antibody B1 CDR H1 is SEQ ID NO: 4, CDR H2 is SEQ ID NO: 5, CDR H3 is SEQ ID NO: 43. CDR L1 is SEQ ID NO: 7, CDR L2 is SEQ ID NO: 8, CDR L3 is SEQ ID NO: 9. The VH is SEQ ID NO: 44 and the VL is SEQ ID NO: 25 or 26.
Antibody B6 CDR H1 is SEQ ID NO: 4, CDR H2 is SEQ ID NO: 5, CDR H3 is SEQ ID NO: 46. CDR L1 is SEQ ID NO: 7, CDR L2 is SEQ ID NO: 8, CDR L3 is SEQ ID NO: 9. The VH is SEQ ID NO: 47 and the VL is SEQ ID NO: 25 or 26.
Antibody D1 CDR H1 is SEQ ID NO: 4, CDR H2 is SEQ ID NO: 5, CDR H3 is SEQ ID NO: 49. CDR L1 is SEQ ID NO: 7, CDR L2 is SEQ ID NO: 8, CDR L3 is SEQ ID NO: 9. The VH is SEQ ID NO: 50 and the VL is SEQ ID NO: 25 or 26.
Antibody C3 CDR H1 is SEQ ID NO: 4, CDR H2 is SEQ ID NO: 5, CDR H3 is SEQ ID NO: 6.
   CDR L1 is SEQ ID NO: 7, CDR L2 is SEQ ID NO: 8, CDR L3 is SEQ ID NO: 9. The VH is SEQ ID NO: 52 and the VL is SEQ ID NO: 25 or 26.
Antibody D4 CDR H1 is SEQ ID NO: 4, CDR H2 is SEQ ID NO: 5, CDR H3 is SEQ ID NO: 53. CDR L1 is SEQ ID NO: 7, CDR L2 is SEQ ID NO: 8, CDR L3 is SEQ ID NO: 9. The VH is SEQ ID NO: 54 and the VL is SEQ ID NO: 25 or 26.
Antibody A4 CDR H1 is SEQ ID NO: 4, CDR H2 is SEQ ID NO: 5, CDR H3 is SEQ ID NO: 15. CDR L1 is SEQ ID NO: 7, CDR L2 is SEQ ID NO: 8, CDR L3 is SEQ ID NO: 9. The VH is SEQ ID NO: 20 and the VL is SEQ ID NO: 25 or 26.
Antibody B3 CDR H1 is SEQ ID NO: 4, CDR H2 is SEQ ID NO: 5, CDR H3 is SEQ ID NO: 21. CDR L1 is SEQ ID NO: 7, CDR L2 is SEQ ID NO: 8, CDR L3 is SEQ ID NO: 9. The VH is SEQ ID NO: 24 and the VL is SEQ ID NO: 25 or 26.
Antibody E1 CDR H1 is SEQ ID NO: 4, CDR H2 is SEQ ID NO: 5, CDR H3 is SEQ ID NO: 45. CDR L1 is SEQ ID NO: 7, CDR L2 is SEQ ID NO: 8, CDR L3 is SEQ ID NO: 9. The VH is SEQ ID NO: 24 and the VL is SEQ ID NO: 25 or 26.
Antibody F5 CDR H1 is SEQ ID NO: 4, CDR H2 is SEQ ID NO: 5, CDR H3 is SEQ ID NO: 51. CDR L1 is SEQ ID NO: 7, CDR L2 is SEQ ID NO: 8, CDR L3 is SEQ ID NO: 9. The VH is SEQ ID NO: 55 and the VL is SEQ ID NO: 25 or 26.

### 2A5 Antibody Family

Antibody 2A5 CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 17. The VH is SEQ ID NO: 14 and the VL is SEQ ID NO: 18 or 19.
Antibody E3 CDR H1 is SEQ ID NO: 11, CDR H2 is SEQ ID NO: 12, CDR H3 is SEQ ID NO: 56. CDR L1 is SEQ ID NO: 7, CDR L2 is SEQ ID NO: 16, CDR L3 is SEQ ID NO: 17. The VH is SEQ ID NO: 57 and the VL is SEQ ID NO: 18 or 19.
Antibody D6 CDR H1 is SEQ ID NO: 11, CDR H2 is SEQ ID NO: 12, CDR H3 is SEQ ID NO: 58. CDR L1 is SEQ ID NO: 7, CDR L2 is SEQ ID NO: 16, CDR L3 is SEQ ID NO: 17. The VH is SEQ ID NO: 59 and the VL is SEQ ID NO: 18 or 19.
Antibody H4 CDR H1 is SEQ ID NO: 11, CDR H2 is SEQ ID NO: 12, CDR H3 is SEQ ID NO: 60. CDR L1 is SEQ ID NO: 7, CDR L2 is SEQ ID NO: 16, CDR L3 is SEQ ID NO: 17. The VH is SEQ ID NO: 61 and the VL is SEQ ID NO: 18 or 19.
Antibody A4 CDR H1 is SEQ ID NO: 11, CDR H2 is SEQ ID NO: 12, CDR H3 is SEQ ID NO: 62. CDR L1 is SEQ ID NO: 7, CDR L2 is SEQ ID NO: 16, CDR L3 is SEQ ID NO: 17. The VH is SEQ ID NO: 63 and the VL is SEQ ID NO: 18 or 19.
Antibody F1 CDR H1 is SEQ ID NO: 11, CDR H2 is SEQ ID NO: 12, CDR H3 is SEQ ID NO: 64. CDR L1 is SEQ ID NO: 7, CDR L2 is SEQ ID NO: 16, CDR L3 is SEQ ID NO: 17. The VH is SEQ ID NO: 65 and the VL is SEQ ID NO: 18 or 19.
Antibody G2 CDR H1 is SEQ ID NO: 11, CDR H2 is SEQ ID NO: 12, CDR H3 is SEQ ID NO: 66. CDR L1 is SEQ ID NO: 7, CDR L2 is SEQ ID NO: 16, CDR L3 is SEQ ID NO: 17. The VH is SEQ ID NO: 67 and the VL is SEQ ID NO: 18 or 19.
Antibody F6 CDR H1 is SEQ ID NO: 11, CDR H2 is SEQ ID NO: 12, CDR H3 is SEQ ID NO: 68. CDR L1 is SEQ ID NO: 7, CDR L2 is SEQ ID NO: 16, CDR L3 is SEQ ID NO: 17. The VH is SEQ ID NO: 69 and the VL is SEQ ID NO: 18 or 19.
Antibody A12 CDR H1 is SEQ ID NO: 11, CDR H2 is SEQ ID NO: 12, CDR H3 is SEQ ID NO: 70. CDR L1 is SEQ ID NO: 7, CDR L2 is SEQ ID NO: 16, CDR L3 is SEQ ID NO: 17. The VH is SEQ ID NO: 71 and the VL is SEQ ID NO: 18 or 19.
Antibody C09 CDR H1 is SEQ ID NO: 11, CDR H2 is SEQ ID NO: 12, CDR H3 is SEQ ID NO: 72. CDR L1 is SEQ ID NO: 7, CDR L2 is SEQ ID NO: 16, CDR L3 is SEQ ID NO: 17. The VH is SEQ ID NO: 73 and the VL is SEQ ID NO: 18 or 19.
Antibody H10 CDR H1 is SEQ ID NO: 11, CDR H2 is SEQ ID NO: 12, CDR H3 is SEQ ID NO: 74. CDR L1 is SEQ ID NO: 7, CDR L2 is SEQ ID NO: 16, CDR L3 is SEQ ID NO: 17. The VH is SEQ ID NO: 75 and the VL is SEQ ID NO: 18 or 19.
Antibody C11 CDR H1 is SEQ ID NO: 11, CDR H2 is SEQ ID NO: 12, CDR H3 is SEQ ID NO: 76. CDR L1 is SEQ ID NO: 7, CDR L2 is SEQ ID NO: 16, CDR L3 is SEQ ID NO: 17. The VH is SEQ ID NO: 77 and the VL is SEQ ID NO: 18 or 19.
Antibody D3 CDR H1 is SEQ ID NO: 11, CDR H2 is SEQ ID NO: 12, CDR H3 is SEQ ID NO: 78. CDR L1 is SEQ ID NO: 7, CDR L2 is SEQ ID NO: 16, CDR L3 is SEQ ID NO: 17. The VH is SEQ ID NO: 79 and the VL is SEQ ID NO: 18 or 19.
Antibody C6 CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 80. The VH is SEQ ID NO: 14 and the VL is SEQ ID NO: 81 or 82.
Antibody H5 CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 83. The VH is SEQ ID NO: 14 and the VL is SEQ ID NO: 84 or 85.
Antibody F3 CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 86. The VH is SEQ ID NO: 14 and the VL is SEQ ID NO: 87 or 88.
Antibody C1 CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 89. The VH is SEQ ID NO: 14 and the VL is SEQ ID NO: 90 or 91.
Antibody C2 CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 92. The VH is SEQ ID NO: 14 and the VL is SEQ ID NO: 93 or 94.
Antibody F4 CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 95. The VH is SEQ ID NO: 14 and the VL is SEQ ID NO: 96 or 97.
Antibody C3 CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 98. The VH is SEQ ID NO: 14 and the VL is SEQ ID NO: 99 or 100.
Antibody E11 CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 101 or 102. The VH is SEQ ID NO: 14 and the VL is SEQ ID NO: 103 or 104.
Antibody A12 CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 105. The VH is SEQ ID NO: 14 and the VL is SEQ ID NO: 106 or 107.
Antibody D11 CDR H1 is SEQ ID NO: 11, CDR H2 is SEQ ID NO: 12, CDR H3 is SEQ ID NO: 13. CDR L1 is SEQ ID NO: 7, CDR L2 is SEQ ID NO: 16, CDR L3 is SEQ ID NO: 108. The VH is SEQ ID NO: 14 and the VL is SEQ ID NO: 109 or 110.

### EXAMPLES

### Example 1—Generation of Purified Tenascin-c FBG as Antigen and Assay Reagents

Purified soluble proteins containing the FBG domain of tenascin-C (TNC FBG) were generated for use as antigens in antibody selections and as reagents in subsequent screening and characterisation assays. To enable selection strategies for isolation of antibodies that bind tenascin-C of multiple mammalian species, a range of DNA expression constructs were synthesised, which incorporated the TNC FBG domain of either human, mouse, rat or dog. A human tenascin-R FBG construct was also prepared for identification of antibodies that displayed unwanted binding to this homologue. Constructs were produced as 6His-tagged proteins with either a rat CD4 or human IgG1 Fc tag coupled to either a C- or N-terminal FBG domain as described below.

### Protein Expression Constructs

All synthetic DNA constructs for antigen expression were synthesised and sequence confirmed by Genscript (Piscataway, USA). FBG domains were cloned into the mammalian expression vectors pBIOCAM4 or BIOCAM5, which fuse the expressed domains with either a rat Cd4 (domains 3 and 4) tag (Chapple et al, 2006) or a human IgG1 Fc tag (Falk et al, 2012) respectively. The vectors were modified from the pCMV/myc/ER plasmid (Invitrogen) (Falk et al, 2012), which contains an endoplasmic reticulum (ER) signal sequence derived from the mouse VH chain, for secretion of expressed proteins. For all constructs which resulted in an N-terminal FBG (e.g. FBG-Fc-His or FBG-rCd4-His) the digested PCR products were ligated with NcoI/NotI cut pBIOCAM4 or pBIOCAM5 vectors. For all constructs which resulted in a C-terminal FBG (e.g. Fc-His-FBG or rCd4-His-FBG), digested PCR products were ligated with BamHI/HindIII cut pBIOCAM4 or pBIOCAM5 vectors. The primers used to amplify the FBG domains are listed in FIG. 10. All constructs were sequence confirmed. To facilitate ELISA screening, an insert encoding a His-tag (primers 2574 and 2575) was cloned between the BamHI and HindIII sites (replacing the His-FLAG tag) for the expression plasmid with a FBG-X (N-terminal FBG) fusion. Full length tenascin C was cloned directly from the Genscript pUC57 plasmid by digestion with BstXI and BamHI and cloned into the BstXI/BamHI cut expression vector pFBG-Fc-His6. To create His-FBG constructs, primers were designed to PCR from an rCd4-His-FBG expression plasmid and the PCR product, encoding His-FBG, was digested with XhoI and HindIII and cloned into the XhoI/HindIII digested pBIOCAM5.

### Protein Expression and Cell Culture

Transfection quality plasmid DNA was prepared using the Machery Nagel Nucleobond Xtra Midi kit (740410.50, Fisher Scientific, UK). HEK293F suspension cells and Freestyle media, for antigen and antibody expression, and RPMI media were from Life Technologies (Paisley, UK). Transfection of HEK293F cells was carried out as described previously (Chapple et al, 2006).

### Protein Purification and QC

Protein affinity purification employed either Ni-NTA agarose or immobilised recombinant protein A resin.

For purification of His-tagged proteins, culture supernatants were mixed with Ni-NTA agarose (1018240, Qiagen, Crawley, UK) for 1 h and the resin transferred to Proteus 1-step midi spin columns (Generon, UK) for centrifugation (200×g, 2 min). Unbound proteins were washed out with phosphate buffered saline (PBS) supplemented with 20 mM imidazole (pH 8). Bound proteins were eluted in fractions through addition of 300 mM imidazole in PBS (pH 8) and column centrifugation (200×g, 2 min). Pooled fractions containing eluted protein were placed in Gebaflex Midi dialysis tubes (Generon D010; molecular weight cut-off 3.5 kDa) and dialysed against PBS.

Fc-tagged proteins and antibodies expressed as human IgG4 were purified using protein A sepharose (PC-A25, Generon, Maidenhead, UK). Culture supernatants were clarified by centrifugation (2500×g, 15 min) and mixed with protein A sepharose overnight at 4° C. before transfer of the resin to Proteus 1-step midi spin columns (Generon, UK). Columns were centrifuged (200×g, 2 min) and washed with PBS to remove unbound protein. Fc-tagged or IgG4 proteins were eluted in fractions from the protein A with 0.2 M glycine (pH 2.8) into Tris-HCl (pH 8) by centrifugation (200×g, 2 min). Eluted fractions were pooled and dialysed against PBS in Gebaflex Maxi dialysis tubes (Generon D045; molecular weight cut-off 8 kDa).

Proteins were analysed for purity and concentration by SDS-PAGE (4-12% gel) and spectrophotometry (0D280 using theoretical extinction coefficient). Where purified proteins were used in cell-based assays the endotoxin content was first determined by limulus amoebocyte lysate chromogenic endotoxin assay (Pierce). Proteins were not used if endotoxin levels exceeded 1 endotoxin unit per milligram (i.e. 1 EU/mg).

### Example 2—Isolation of Primary Anti-FBG Antibodies Antibody Phage Display

Antibodies against tenascin-C FBG domain were isolated using the lontas Ltd proprietary human antibody phage display library, which was constructed using DNA isolated from 43 human lymphocyte donors. Selections, phage rescues and subcloning into pSANG10 (Martin et al, 2006) were all performed as described previously (Schofield et al, 2007) using techniques that are well known in the art.

Two rounds of panning selections were performed on immobilised TNC FBG fused to human IgG1 Fc or rCd4 at either the N terminus of the fusion partner (e.g. FBG-Fc, FBG-rCd4) or at the C terminus (Fc-FBG, rCd4-FBG). Phage antibody libraries containing either kappa (κ) or lambda (λ) variable light chains (V_{L}) were panned separately to facilitate later sub-cloning to Fab expression vectors containing either constant light (C_{L}) kappa (κ) or lambda (λ) chains.

Polyclonal phage populations were prepared from the selected populations and were tested in ELISA (polyclonal phage ELISA) using ELISA plates coated with TNC FBG antigen or appropriate fusion partner (Fc or rCd4). After incubation with phage, plates were washed, and bound phage detected using peroxidase-conjugated anti-M13 antibodies. Enrichment of antigen-specific binders between rounds 1 and 2 of selection and a greater proportion of FBG binders compared to anti-Fc or -rCd4 phage in the round 2 output populations, indicating that the selections were successful.

Confirmation of scFv Binding to Antigen and Cross-Reactivity Assay by ELISA

Round 2 selection outputs were expressed as individual scFv clones to confirm antigen recognition in ELISA binding assays. Output populations were sub-cloned into the bacterial expression vector pSANG10 (Martin et al, 2006), transformed into E. coli BL21 (DE3), and individual transformants were induced in 96-well plates as described previously (Schofield et al, 2007). E. coli supernatants were collected and assayed for binding of scFv to TNC FBG using DELFIA-based ELISA, using europium-labelled anti-FLAG detection antibodies. The most successful selections with the A library were based on panning against the antigens rCd4-FBG and Fc-FBG (selections 147 and 148). For the κ library, the most successful selections were obtained with the antigens FBG-rCd4 (150), rCd4-FBG (152) and Fc-FBG (153). The 79 positive clones from this ELISA screen were selected for further analysis.

Cross-reactivity ELISA showed that 67/79 (85%) of anti-human FBG scFv were cross-reactive to mouse TNC FBG. DNA sequence analysis of the anti-FBG scFv indicated excellent sequence diversity. For example, selections 147 and 148 from the V_{L} λ library contained 92% unique variable heavy (V_{H}) complementarity determining region 3 (CDR3) sequences, and selections 150, 152 and 153 from the VL κ library contained 67%, 91% and 100% unique variable V_{H} CDR3 sequences, respectively.

A further 1425 clones isolated from the most effective selections were screened by ELISA and this resulted in the identification of an additional 401 scFv with FBG-binding specificity. These clones, together with the 79 scFv identified in initial ELISAs were chosen for further evaluation.

The 1425 clones were further tested in a specificity ELISA in which each scFv was tested for binding to human Tenascin R FBG and also to human, mouse, rat and dog TNC FBG. Clones were ranked according to the ELISA signal obtained for binding to Tenascin C divided by the signal for Tenascin R FBG binding. The top 250 clones with a ratio above 50 were taken for subcloning and further analysis.

### Example 3—Screening of Primary Anti-FBG Antibodies in a Functional Assay

Anti-FBG scFv were reformatted either as bivalent scFv-Fc or as monomeric Fabs for evaluation of their activity as inhibitors of FBG-evoked signalling in a whole cell assay system.

The top 50 anti-FBG scFv, ranked by primary ELISA signal, for each of the selections 147, 148, 150, 152 and 153 were sub-cloned into the mammalian expression plasmid pBIOCAM5 (Falk et al, 2012) as individual selection populations and expressed by transient transfection in HEK293F cells (Chapple et al, 2006). For Fab expression, pooled A or κ scFv variable heavy (V_{H}) and variable light (V_{L}) inserts were cloned into a dual promoter Fab expression vector (pFab-dual-κ or pFab-dual-A, depending on the light chain germ-line) using a proprietary lontas Ltd protocol. Culture supernatants were screened for activity in the THP-1 cell assay and selected scFV-Fc and Fab hits were affinity purified for re-assaying and confirmation of inhibitory activity.

### THP1-Blue™ Reporter Cell Assay

Tenascin-C has been shown to elicit the generation of cytokines in inflammatory cells and fibroblasts by interaction of the FBG domain with cellular TLR4 (Midwood et al, 2009). The receptor signalling cascade leading to generation of inflammatory cytokines such as TNFa, IL-8 and IL-6 involves activation of the transcription factor NF-κB. This process can be studied in ‘reporter’ cell lines modified to respond to NF-κB activation with generation of an easily measured protein signal. The THP1-Blue™ reporter cell line (InvivoGen; Toulouse, France) is derived from the human THP-1 monocyte cell line and stably expresses an NF-κB-inducible secreted alkaline phosphatase (SEAP) reporter construct. These cells also constitutively express cell surface TLR4, which enables the signalling activity of TNC FBG fusion proteins to be readily measured using colorimetric or fluorimetric quantitation of SEAP in culture supernatants using medium- to high-throughput assay methods.

Activity at low FBG concentrations is critical to the success of any screening assay; if the concentrations of FBG required to produce a robust increase in the reporter protein are too high then the expression levels and concentrations of scFv, Fc-ScFv or Fab constructs required to fully inhibit any such signal would be unacceptable for a screen. Fc-FBG produces a robust SEAP signal at low nM levels in this cell assay (CD4-FBG did not produce a response in this concentration range).

THP1-Blue™ cells were cultured and passaged in supplemented RPMI media according to supplier's protocols (http://www.invivogen.com/PDF/THP1_Blue_NF_kB_TDS.pdf), except that cells were grown in ultra-low attachment T75 flasks. For assays, THP1-Blue™ cells were added to 96-well tissue culture plates (100,000 cells/well) containing Fc-FBG (3 or 10 nM) in RPMI medium in a total volume of 170 μl. Culture supernatants containing expressed scFv-Fc or Fab, or affinity purified antibody in PBS, was added in a volume of 30 μl and cells were incubated for 18 h at 37° C. Supernatants were harvested and assayed for either SEAP using the Attophos AP fluorimetric quantitation system (S1000; Promega) or IL-8 content using the DuoSet ELISA development system (DY208; R&D Systems, UK) according to the supplier's instructions. Data were plotted and curves fitted using Prism software (GraphPad).

Screening of anti-FBG antibodies as HEK293F culture supernatants highlighted putative inhibitors of Fc-His-FBG evoked signalling in THP1-Blue™ cells of which 9 were confirmed when re-assayed as purified scFv-Fc or Fab. Fc-His-FBG is key to having the potency assays work. Monomeric FBG does not elicit any cytokine response in THP-1Blue and human cells.

### Example 4—Functional Characterisation of Primary Anti-FBG Antibodies

### ELISA Cross-Reactivity Assays

The panel of 9 human FBG signalling inhibitors identified in the THP1-Blue™ functional assay was evaluated by ELISA for cross-reactivity to rat, mouse, and dog FBG. Binding to the human tenascin-R FBG homologue was also determined. Assay wells were coated with human, rat, mouse, and dog TNC FBG-rCD4, or human TNR FBG-rCd4 fusion proteins and binding of Fabs was detected using anti-kappa or anti-lambda mAb followed by Europium-conjugated anti-mouse mAb. ELISA results revealed that the C3 antibody showed good cross-reactivity to other mammalian homologues of human TNC FBG, with lower apparent binding to human TNR FBG. These were:

### Determination of Binding Affinity by Surface Plasmon Resonance

The affinity and association and dissociation kinetics of selected Fabs for binding to the human, rat and mouse TNC FBG, and human TNR FBG were measured by surface plasmon resonance (SPR) at 25° C. Experiments were performed using a BIAcore T100 instrument with CM5 sensor chip according to the protocol provided with the Human Fab Capture Kit (GE, 28-9583-25). Varying concentrations of rCd4-FBG were injected into a flow-cell with immobilised Fab and a reference flow-cell. After reference signal subtraction, the data was fitted to a global 1:1 fit using theT100 BIAevaluation software.

The calculated kinetic constants are shown in Table 3. The rank order of affinity of Fabs for human TNC FBG was B12 (110 pM)>. All Fabs displayed low nanomolar affinity for rodent TNC FBG, and affinities for human TNR FBG were typically greater than 60-fold lower than human TNR FBG.

### Inhibitory Potency Assays

The potency of purified Fabs for neutralisation of huFc-His-FBG activity was determined in the THP1-Blue™ assay, using measures of TLR4-mediated secreted alkaline phosphatase and IL-8 cytokine production. Assays were conducted as described in Example 2, except that purified Fabs were added to assay wells at a range of concentrations (0.3-100 nM) to enable calculation of IC₅₀ values using Prism software (GraphPad).

The C3 antibody of the present disclosure is derived from an antibody referred to as B12.

| | | Kinetics | | | |
|---|---|---|---|---|---|
| | | K_{D} | Kₐ | K_{d} | Steady |
| Fab | FBG | (nM) | (M⁻¹s⁻¹) × 10⁵ | (s⁻¹) × 10⁻⁴ | State |
| B12 | Hu TNC | 0.111 | 26.62 | 3.0 | N/A |
| | Mu TNC | 13 | 52.15 | 675.5 | 18.7 |
| | Rat TNC | 7.9 | 94.59 | 747.9 | N/A |
| | Hu TNR | 33.9 | 13.96 | 472.5 | 36.1 |

Anti-FBG Fab binding kinetic data determined by surface plasmon resonance (SPR) spectroscopy. K_{D}, equilibrium dissociation constant; Kₐ, association constant; Kₐ, dissociation constant

### Example 5—Generation and Isolation of Optimised Antibodies to huTNC FBG Domain Affinity Maturation by Targeted CDR Mutagenesis

Anti-FBG antibody B12 was selected for affinity maturation. Targeted CDR mutagenesis was carried out by randomising VH and VL CDR3 residues in blocks of 6 amino acids using Kunkel mutagenesis (Fellouse and Sidhu, 2007; Kunkel et al., 1987; Sidhu and Weiss, 2004). Due to the longer VH CDR3s (10-16 residues) for the given clones randomisation was done in three overlapping blocks and the VL CDR3s (9 residues) were randomised in two overlapping blocks. Randomisations were carried out using NNS (N=A/G/C/T and S=G/C) degenerate primers that could encode any of the 20 amino acids (and only a single amber stop codon) at a given position from 32 codon combinations. The following library was created.

| Library | Sub library | Size | Combined size |
|---|---|---|---|
| B12 VH | B12 VH 3.1 | 1.8 × 10⁹ | 6.1 × 10⁹ |
| | B12 VH 3.2 | 1.6 × 10⁹ | |
| | B12 VH 3.3 | 1.7 × 10⁹ | |
| B12 VL | B12 VL 3.1 | 2.6 × 10⁹ | 7.7 × 10⁹ |
| | B12 VL 3.2 | 5.1 × 10⁹ | |

Estimated sizes of the CDR3 randomised libraries

### High Stringency Phage Display Selections

Phage-antibody selections on streptavidin Dynabeads were performed as described previously (Dyson et al, 2011). Multiple rounds of solution-phase selections were carried out on biotinylated rCd4-His-FBG to enrich for affinity improved clones. The optimum antigen concentrations for each round were determined empirically by selecting against a range of antigen concentrations and comparing the output numbers with a no-antigen control. The stringency of selection was increased by reducing the amount of antigen used in each round. No further rounds of selection were carried out after the selection window (the fold difference between phage titres from selection outputs and no antigen control) dropped below 10. Hence, three rounds of selection were carried out on biotinylated human rCd4-His-FBG for all libraries except B12 which was subjected to a fourth round of selection due to the large selection windows observed at round 3. All libraries were subjected to deselection against streptavidin beads and tenascin-R (100 nM for rounds 1 to 3 and 1 nM for round 4) at each round of selection to avoid unwanted cross reactivity to streptavidin or tenascin-R. In addition, a hybrid selection strategy in which the human and mouse antigens were alternated between rounds of selection was performed for the B12 randomised libraries only. The reason for performing this extra selection on the B12 libraries was the large difference in affinity observed for the B12 parental antibody binding to human and mouse rCd4-his-FBG. Furthermore, an additional round of selection was carried out to select for antibody clones with superior off-rates. In off-rate selections, phage were allowed to bind to the biotinylated antigen (1 nM in this case), and a large excess of non-biotinylated antigen (500 nM) was subsequently added to the reaction and incubated for 20 h or 40 h. The non-biotinylated antigen serves as a competitor and captures the phage antibodies that dissociate from the biotinylated antigen, i.e. only the antibodies with longer off-rates will be recovered at the end of the selection (Hawkins et al., 1992; Zahnd et al., 2010). The output phage titres for each round of selection together with calculated selection windows are shown in Tables below.

| | | | | Selection | Selection |
|---|---|---|---|---|---|
| CDR3 | | | | window for | window for |
| randomised | 10 nM | 1 nM | 0 nM | 10 nM | 1 nM |
| libraries | Selection | Selection | Selection | selection | selection |
| B12 VH | 6 × 10⁷ | 2.6 × 10⁷ | 1 × 10⁵ | 600 | 260 |
| B12 VL | 6 × 10⁷ | 5 × 10⁷ | 2 × 10⁵ | 300 | 250 |

Selection output titres. Round 1 selections. Phage output titres were determined as described previously (Schofield et al, 2007)

| | | | | Selection | Selection |
|---|---|---|---|---|---|
| CDR3 | | | | window for | window for |
| randomised | 200 pM | 50 pM | 0 nM | 200 pM | 50 pM |
| libraries | Selection | Selection | Selection | selection | selection |
| B12 VH | 1 × 10⁸ | 6.75 × 10⁷ | 2 × 10⁴ | 5000 | 3375 |
| B12 VL | 1.2 × 10⁸ | 8.1 × 10⁷ | 4 × 10⁴ | 3000 | 2025 |
| B12 VH on mu | 7 × 10⁶ | | 2 × 10⁴ | 350 | |
| TNC FBG | | | | | |
| B12 VL on mu | 7.5 × 10⁶ | | 4 × 10⁴ | 187 | |
| TNC FBG | | | | | |

Selection output titres. Round 2 selections. Phage output titres were determined as described previously (Schofield et al, 2007)

| | | | | Selection | Selection |
|---|---|---|---|---|---|
| CDR3 | | | | window for | window for |
| randomised | 5 pM | 1 pM | 0 nM | 5 pM | 1 pM |
| libraries | Selection | Selection | Selection | selection | selection |
| B12 VH | 1.5 × 10⁷ | 4 × 10⁶ | <1 × 10⁵ | >150 | >40 |
| B12 VL | 2.7 × 10⁷ | 3.5 × 10⁶ | <1 × 10⁵ | >270 | >35 |
| Hybrid | | | | Selection | Selection |
| selections on | | | | window for | window for |
| B12 libraries | 20 pM | 5 pM | 0 pM | 20 pM | 5 pM |
| (Hu-mu-hu) | Selection | Selection | Selection | selection | selection |
| B12 VH | 1 × 10⁸ | 7.7 × 10⁶ | <1 × 10⁵ | >1000 | >77 |
| B12 VL | 1.3 × 10⁸ | 1.8 × 10⁷ | <1 × 10⁵ | >1300 | >78 |

Selection output titres. Round 3 selections. Phage output titres were determined as described previously (Schofield et al, 2007)

### ELISA Screen

An anti-FLAG capture ELISA was performed to screen for clones that had an improved affinity for mouse FBG binding compared with the parental antibodies.

E. coli clones harbouring scFv pSANG10 expression plasmids were induced in 96-well plates with auto-induction media as described previously (Schofield et al, 2007). E. coli supernatants were harvested for ELISA assays. ELISA used the DELFIA (dissociation enhanced lanthanide fluorescent immunoassay) system with Europium-labelled anti-FLAG antibody (Sigma, Aldrich, UK). Black immunosorb plates (Nunc) were coated overnight with anti-FLAG M2 antibody (Sigma, F3165, 5 μg/ml in PBS, 50 μl per well), in wells blocked by the addition of 2% milk powder, PBS (PBS-M, 300 μl per well). Plates were washed three times with PBS-T (PBS, 0.1% Tween-20) and three times with PBS followed by the addition of a 1:2 dilution of 96-well auto-induction culture supernatants containing expressed scFv in PBS-M (50 μl per well). The plates were incubated for 1 h, washed as above and biotinylated mouse or human rCd4-His-FBG (5 μg/ml in PBS-M, 50 μl) added to each well. Plates were incubated for a further 1 h, washed and Strepravidin-Eu added (Perkin Elmer, 1 μg/ml, PBS-M, 50 μl), incubated for 30 min, washed and DELFIA enhancement solution added (50 μl) and plates read on a Perkin Elmer Fusion plate reader (excitation=320 nm, emission 620 nm). The format of the assay is shown in.

In this assay differences in scFv expression level are normalised because the expression levels of scFv in auto-induction cultures saturate the anti-FLAG coated wells. Therefore, the signals obtained in the assay reflect the amount of biotinylated rCd4-His-FBG bound after washing, which will be a function of the off-rate of that clone for mouse or human FBG. ELISA screening of the selection output from the B12 sub-library revealed clones with significantly improved binding to mouse TNC FBG.

### HTRF Screen

An HTRF-based competition assay was developed to screen for antibody variants with improved binding to human TNC FBG.

All samples and reagents were prepared in assay buffer (50 mM NaPO₄, 0.1% BSA, 0.4 M KF, pH 7.0) at 4× the stated concentration. 5 μl of each reagent was subsequently added to low volume 384-well assay plates (Greiner, 784075) to give a final reaction volume of 20 μl. IgG antibodies were labelled using the d2 labelling kit (CisBio, 62D2DPEA) as directed by the manufacturer. Streptavidin europium cryptate (CisBio, 610SAKLA, Lot#25C) was used at a final concentration of 1.8 ng active moiety (SA) per 20 μl reaction as recommended by the manufacturer. Biotinylated rCd4-His-FBG was prepared using EZ-link Sulfo-NHS-LC-Biotin reagent (Thermo Scientific, 21327) the extent of biotinylation was quantified using biotinylation fluorescence quantitation kit (Thermo Scientific, 46610). Where appropriate, supernatants containing scFv (prepared as described above for ELISA assays) were added to the 384-well assay plate at a final dilution of 1/20 (i.e. 1/5 dilution in assay buffer followed by addition of 5 μl diluted sample to the 20 μl FRET assay). The concentrations of d2-labelled B12 IgG used for screening were 1.25 nM. Unless otherwise stated, biotinylated rCd4-His-FBG (biotin:protein ratio=1.8:1) was present at either 2.2 nM (in assays using the 2A5 IgG antibody) or 1 nM (in experiments using B12 IgG). Samples were incubated for approximately 1 h at room temperature and the FRET signal was determined using a BMG Pherastar instrument: excitation=320 nm; emission=620 nm and 665 nm; integration start time=60 μs; integration time=500 μs; 100 flashes per well. For competition assays containing culture supernatant, biotinylated rCd4-His-FBG antigen was pre-incubated with streptavidin europium cryptate for 45 min prior to addition of reagents to the assay plate. All FRET signals are presented as ΔR, where R=(E665/E620×104) and ΔR=(Rsample−Rbackground fluorescence).

Culture supernatants containing unlabelled scFv clones from affinity selected mutant libraries were tested for inhibition of the interaction between FBG and the fluorophore-labelled parental IgG antibody. The relative ranking of clones exhibiting FRET signals within the useful range in both assays was broadly unchanged, indicating that they were competing for similar epitopes. Hence, all B12 scFv variants from affinity maturation selections were screened for their ability to inhibit the binding of B12 IgG molecules to human TNC FBG. The parental clones, expressed as scFvs in parallel with the affinity matured clones, were used as benchmarks.

ScFv were sequenced and a panel of clones with unique VH or VL CDR3 sequences was selected for further study in human IgG4 format, based on their binding to mouse and human TNC FBG in the ELISA and HTRF assays. respectively.

| | | | | | | | | | | % | |
|---|---|---|---|---|---|---|---|---|---|---|---|
| | | | | | | | | | | inhibition | |
| | | Total | % inhibition of FRET signal | | | | | | | by parent | |
| CDR3 | Selection | clones | 0- | 25- | 51- | 76- | 86- | 91- | | scFv | |
| Library | type | tested | 25% | 50% | 75% | 85% | 90% | 95% | ≥96% | 2A5 | B12 |
| B12 | 100 fM | 46 | 6 | 2 | 3 | 8 | 5 | 6 | 16 | 19 | 86 |
| VH | | | | | | | | | | | |
| B12 | Hybrid | 46 | 3 | 3 | 5 | 5 | 3 | 9 | 18 | 19 | 86 |
| VH | 5 pM | | | | | | | | | | |

HTRF screen for clones with improved affinity for human rCD4-FBG.

Variants of antibody B12 showed ≥4-fold improvement for mouse FBG binding, and 91% inhibition of HTRF signal. In total, 31 clones fitting these criteria with unique CDR3 sequences were identified below.

| Library | Clone name | CDR sequence |
|---|---|---|
| B12 VH | 165_13_B1 | VMSSMEDAFDI |
| | 165_13_B6 | GQKGEGDTFDI |
| | 165_13_D1 | GTRGEGDTFDI |
| | 165_13_C3 | SYQSDEDAFDI |
| | 165 13 D4 | GTVGEGDTFDI |
| | 165_13_A4 | DKYPVLDTFDI |
| | 165_13_B3 | ALARGHDTFDI |
| | 165_13_E1 | DISAVMDVPQT |
| | 180_11_F5 | VMRTGLDTFDI |

Heavy or light chain CDR3 sequences of clones identified with improved binding to mouse and human TNC FBG and chosen for conversion to human IgG format for further study.

These are heavy or light chain sequences of antibody clones that bind to human and mouse TNC FBG and thus have potential utility in the methods, uses, compositions and compounds of the present invention. For example, antibodies that bind TNF FBG having these CDR3 sequences may be useful in identifying, inhibiting the function of, detecting and purifying TNC or TNC FBG.

### Conversion to IgG4 Format and Determination of Binding Kinetics

The 31 scFv of interest were sub-cloned into a human IgG4 expression vector for generation of antibodies as human IgG4 with a hinge-stabilising mutation (S241P; Angal et al, 1993). IgG4 antibodies were transiently expressed in HEK-293F cells and culture supernatants were screened using surface plasmon resonance spectroscopy for ranking of their off-rates for binding to human and mouse TNC FBG, and human TNR FBG. Briefly, surface plasmon resonance (SPR) experiments were performed using a BIAcore T100 instrument and followed the protocol according to the Human antibody capture kit protocol (GE, BR-1008-39). For off-rate screening, 10,000 response units (RU) of anti-human Fc IgG (GE, BR-1008-39) was immobilised on flow-cells (FC1 and FC2) of a Series 5 CM5 dextran sensor chip (BR-1005-30) using EDC/NHS cross-linking chemistry according to the amine coupling kit protocol (GE, BR-1000-50). Culture supernatants containing expressed IgG4 were diluted 1:2 with 2×PBS-T and injected into FC2 (flowrate 5 μl/min, 60s contact time) to enable antibody capture at 25° C. Antibody capture levels ranged from 308 to 1975 RU depending on the expression level of the antibody in the supernatant. A fixed concentration of antigen (15 nM of human and mouse TNC rCd4-His-FBG and 100 nM of human TNR rCd4-His-FBG) was injected with a flow-path via FC 1 (reference flow cell) and FC 2 (antibody capture flow cell), with a flow rate of 30 μl/min, and the association and dissociation phases measured over 1 and 5 min time periods, respectively. Regeneration of the binding surface employed 3M MgCl₂ with 30s contact time. Off rates were determined by reference cell subtraction and fitting the sensogram experimental data assuming a 1:1 interaction using BIAevaluation software (GE, BR-1005-97). Results of the off-rate screen are summarised in the table below.

| | kd(s⁻¹ × 10⁻⁴) for rCD4-His-FBG | | |
|---|---|---|---|
| Clone name | Human TNC FBG | Mouse TNC FBG | Human TNR FBG |
| 165_13_C3 | 0.00095 | 0.033 | 120 |
| 162_02_C3 | 0.0149 | 20 | 6350 |
| B12 parent | 1.5 | 300 | 1001 |

Surface plasmon resonance screen for ranking of human IgG4 anti-FBG off-rates

Clones were ranked according to low off-rate for human and mouse TNC rCd4-His-FBG, and high-off rate for human TNR rCd4-His-FBG. The 3 highest-ranking antibodies from each library were prioritised for more detailed kinetic analysis as purified IgG4. These clones are shown in table below.

| Clone | VH CDR3 |
|---|---|
| B12 parent | DISAVPDTFDI |
| 165_13_B1 | VMSSMEDAFDI |
| 165_13_D1 | GTRGEGDTFDI |
| 165_13_C3 | SYQSDEDAFDI |

Heavy chain CDR3 amino acid sequences of B12 mutants with improved FBG binding off-rate characteristics

Detailed kinetic parameters were evaluated for the 9 prioritised IgG4 antibodies. Binding characteristics were determined for interaction with human, rat and dog TNC rCD4-His-FBG, and human TNR rCD4-His-FBG. Kinetic assays followed essentially the same protocols as for the off-rate determinations described above, with some modifications as follows. To improve the accuracy of kinetic parameter determination, anti-human Fc IgG was immobilised at lower levels (2229 RU), resulting in a corresponding reduction in the amount of anti-FBG IgG4 captured. Purified anti-FBG IgG4 was diluted to a concentration of 3.5 nM in PBS, pH 7.4, 0.05% Tween-20 and injected into FC2 at a flow rate of 10 μl/min, 60s contact time. This typically resulted in an average of 80 RU of antibody captured (range: 55 RU to 90 RU). Antigens were prepared by doubling dilution in PBS, pH 7.4, 0.05% Tween-20 (highest concentration 100 nM except mouse rCD4-His-FBG which was 7 nM). Assays were performed at 37° C. (30 μl/min, 120s contact time; mouse rCD4-His-FBGFBG 10 μl/min, 60s contact time), with both the flow cell and injection chamber equilibrated to this temperature. As before, kinetic parameters were determined by reference cell subtraction and fitting the sensogram experimental data assuming a 1:1 interaction using BIAevaluation software (GE, BR-1005-97).

All nine antibodies displayed improved binding to mouse TNC FBG domain compared to the non-affinity matured parent clones, and antibodies 165_13_B1, 165_13_C3, and 160_01_A4 exhibited sub-nanomolar K_{D} values for binding to human TNC FBG, with >70-fold lower affinity to the human TNR FBG analogue:

| | | | | |
|---|---|---|---|---|
| Antibody | rCD4-His-FBG | K_{D} | Kₐ | K_{d} |

| IgG4 | Parent | Species | Tenascin | (nM) | (M⁻¹s⁻¹) × 10⁴ | (s⁻¹) × 10⁻⁴ |
|---|---|---|---|---|---|---|
| B12 | B12 | Human | TNC | 0.24 | 47.1 | 11.2 |
| | | Mouse | TNC | 4.5 | 30 | 13.8 |
| 165_13_B1 | B12 | Human | TNC | 0.26 | 72.7 | 18.8 |
| | | Mouse | TNC | 0.96 | 73.3 | 7.06 |
| | | Rat | TNC | 2.20 | 31.1 | 68.4 |
| | | Dog | TNC | 2.85 | 65.5 | 187 |
| | | Human | TNR | 94.4 | 12.2 | 1149 |
| 165_13_C3 | B12 | Human | TNC | 0.072 | 116 | 8.3 |
| | | Mouse | TNC | 0.46 | 97.2 | 4.45 |
| | | Rat | TNC | 1.22 | 38.9 | 47.3 |
| | | Dog | TNC | 1.80 | 59.7 | 108 |
| | | Human | TNR | 35.8 | 12.0 | 431 |

Anti-FBG IgG4 binding kinetic data determined by surface plasmon resonance at 37° C.

### Example 6—Determination of FBG-C Epitope which C3 Antibody Binds to Production of his-TNC-FBG

His-TNC-FBG was cloned and expressed in CHO cells. At the end of the production phase the culture was centrifuged (1000 g, 30 min, 15° C.). A 5 mL HisTrap FF (GE Healthcare) was equilibrated with PBS pH 7.4. After loading the column with supernatant the column was washed with PBS pH 7.4 before being eluted in 0.5 M imidazole in PBS. Eluted protein was loaded onto HiLoad 16/600 Superdex 200 pg and run with 20 mM Na-phosphate, 130 mM NaCl.

The monomeric peak was pooled and the final product was concentrated to 1 mg/mL and stored frozen in small aliquots.

### Production of NSCT-141

NSCT-141 product was cloned, expressed in CHO-cells. The production culture was harvested and clarified supernatant was concentrated by tangential flow filtration before being purified on a Mabselect SuRe column (GE Healthcare). Products were neutralised by the addition of 2×PBS, and adjusted with dilute sodium hydroxide solution to approximately pH 7.2 and stored in aliquots at +5° C.

### Formation of C3 Fab′-Tenascin C Complex

The C165_13_C3* IgG4 antibody was subjected to pepsin cleavage in order to produce a C3 Fab′ antibody. Following pepsin cleavage, the Fab′ was subjected to 2-Mercaptoethylamine-HCl (2-MEA), a mild reductant which selectively reduced the hinge-region disulfide bonds in the Fab′. This was followed by N-Ethylmaleimide (NEM) treatment then forms stable covalent thioether bonds with the reduced cysteine residues, enabling them to be permanently blocked to prevent further disulfide bond formation.

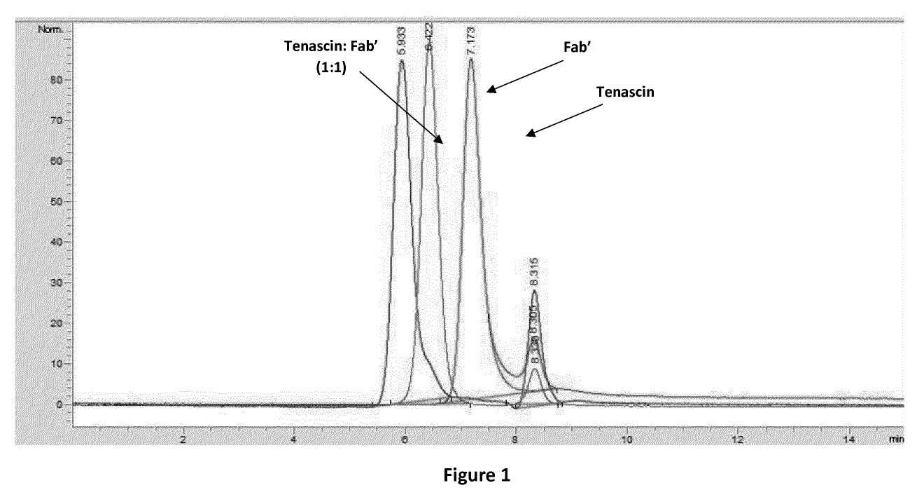

An experiment was conducted to determine the optimal molar ratio for the production of the C3 Fab′-Tenascin C complex. FIG. 1 shows a chromatograph of a SEC analysis which confirmed complex formation at an optimised molar ratio of 1:1.

Based on the results of the trial, 10 mg of Hi-HTNC-FBG peptide (SEQ ID NO: 10) was incubated with 20 mg of the C3 Fab′ for the large scale production of the Fab′-Tenascin complex. The complex was purified by size exclusion chromatography (SEC) using Superdex 200 (GE Healthcare Lifesciences) columns. The purified complex was eluted using a buffer comprising 25 mM Tris pH 7.5, 150 mN NaCl and 1 mM EDTA.

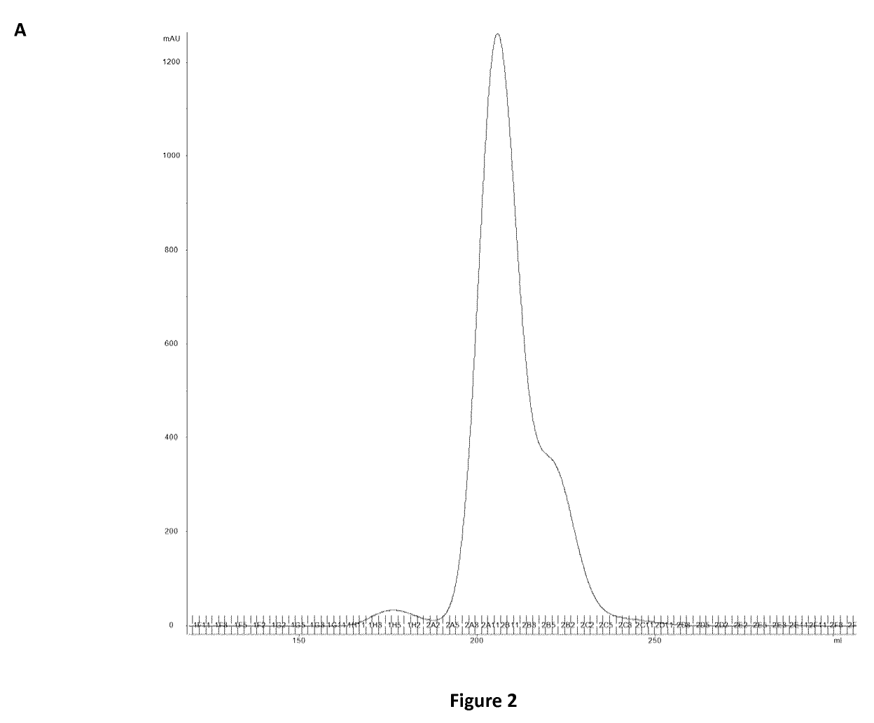

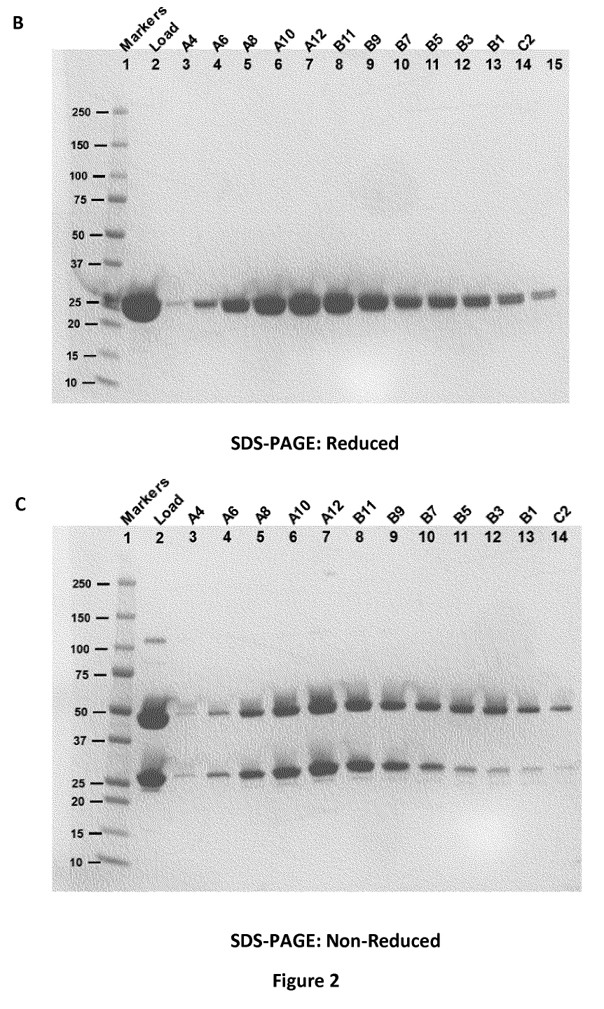

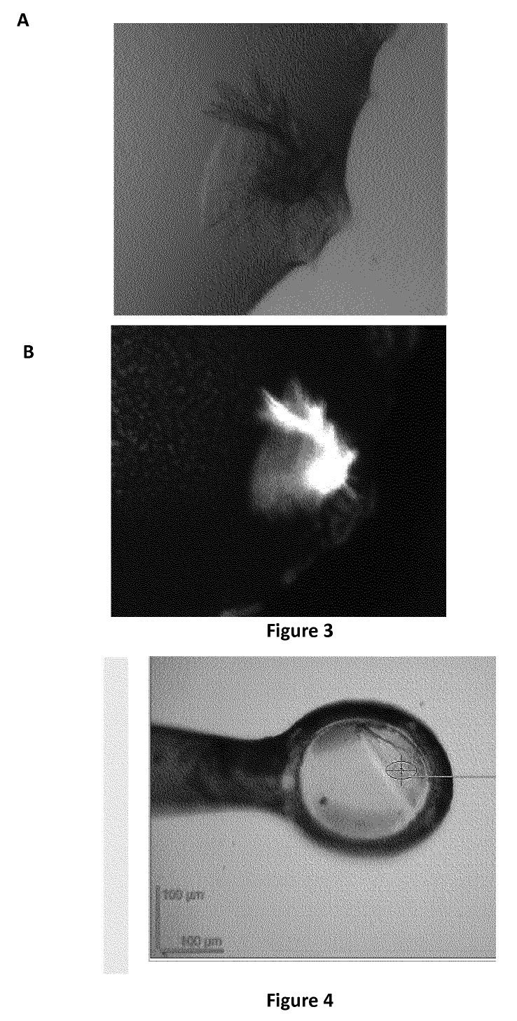

FIG. 2A shows a chromatograph of the Fab′-Tenascin Ccomplex indicating that the eluted sample was substantially pure. The sample was then subjected to SDS-PAGE (see FIGS. 2B and 2C). The bands were of the expected size.

Finally, the Fab′-Tenascin C complex was concentrated up to 38.5 mg/ml before it was used for crystallisation.

### Crystallisation of Fab′-Tenascin Complex

The interaction between C3 Fab′ and Tenascin C was studied by X-ray crystallography. Crystallisation, Structure Determination and Refinement of the crystal structure of Human Tenascin C, Fibrinogen C-terminal domain complexed with C3 Fab′.

### Crystallisation

The human Tenascin C (residues 1974-2201)/C3 Fab′ complex was crystallized in sitting drops through the vapour phase at 22° C. against a reservoir solution of 30 μl containing 100 mM Bis-tris pH 6.5, 200 mM magnesium chloride, 20% PEG 6000 in low profile Swiss-Sci 3 well plates. The drops contained 100 nl of protein at 11.8 mg/ml (based on an estimated Extinction coefficient of 122,000 M⁻¹ cm⁻¹ and MW of 77.4 kDa) in 25 mM Tris pH 7.5, 150 mM sodium chloride, 1 mM EDTA plus 80 nl reservoir solution and 20 nl seed solution, which was required for improved crystal morphology. The crystals belong to the space group P1 2₁ 1 with cell dimensions a=75.8 Å; b=116.9 Å; c=78.7 Å, and α=γ=90°; β=90.4°. Two molecules of the Tenascin C/C3 Fab′ complex are in the asymmetric unit.

### X-Ray Diffraction Collection

Crystals were cryo protected in 20% (v/v) glycerol (diluted in reservoir solution), prior to flash-cooling in liquid nitrogen. X-ray diffraction data was collected from a single crystal on a PILATUS 6M-F at the ID30B beamline station at the European Synchrotron Radiation Facility synchrotron, Grenoble, France. The wavelength of the X-ray beam was 0.9763 Å. The data were processed with XDS and aimless (Kabsch, W. Acta Cryst. D66, 125-132 (2010); Evans, P. R. and Murshudov, G. N. Acta Cryst. D69, 1204-1214 (2013)).

### Structure Determination

The crystal structure of human Tenascin C, Fibrinogen C-terminal domain/C3 Fab′ complex was determined by Molecular Replacement using the program Phaser (McCoy, A. J. et al., J. Appl. Cryst. 40, 658-674 (2007)). Modified versions of PDB entries 4R9J and 5115 were used as starting models for Molecular Replacement. Remodelling, rebuilding and refinement was undertaken using COOT, Buccanner and Refmac respectively (Emsley, P. et al., Acta Cryst D66, 486-501 (2010); Cowtan, K. Acta Cryst D62, 1002-1011 (2006); Murshudov, G. N. et al Acta Cryst D53, 240-255 (1997)). The final resolution was determined as 1.9 Å.

The model of Tenascin C/C3 Fab′ complex encompasses residues 1975 to 2193 of Tenascin C (see SEQ ID NO., Uniprot: P24821), residues 1 to 213 of the light chain of C3 Fab (SEQ ID NO: 3) and residues 1 to 219 of the heavy chain of C3 Fab (SEQ ID NO 2). The R-factor of the model is 0.151, and R-free is 0.197. The r.m.s. deviation from standard geometry is 0.024 Å for bond lengths and 2.13° for bond angles.

### The Epitope

The structure of Tenascin C (residues 1974-2201)/C3 Fab′ reveals the major contact sites between C3 Fab and Tenascin C, and were identified as clustered mainly at the CDR loops of the antibody and on one face of the Tenascin. According to the numbering sequence shown in SEQ ID NO. 1 for the Tenascin C protein, the residues which interact most closely with the CDR region of C3 Fab′ within 3.0 Å, are Ala2115, Asn2118, Ser2131, Arg2147, Asn2148, Cys2149, His2150, Arg2151, Ser2164, His2171, and His2175. Major residues of Tenascin C that contact C3 Fab′ within 3.5 Å are Ala2115, Tyr2116, Asn2118, Ser2131, Tyr2140, Arg2147, Asn2148, Cys2149, His2150, Arg2151, His2163, Ser2164, Phe2170, His2171, His2175 Residues that contact C3 Fab′ within 4.0 Å are Ala2115, Tyr2116, Asn2118, Ser2131, Ile2133, Tyr2140, Arg2147, Asn2148, Cys2149, His2150, Arg2151, His2163, Ser2164, Phe2170, His2171, His2175.

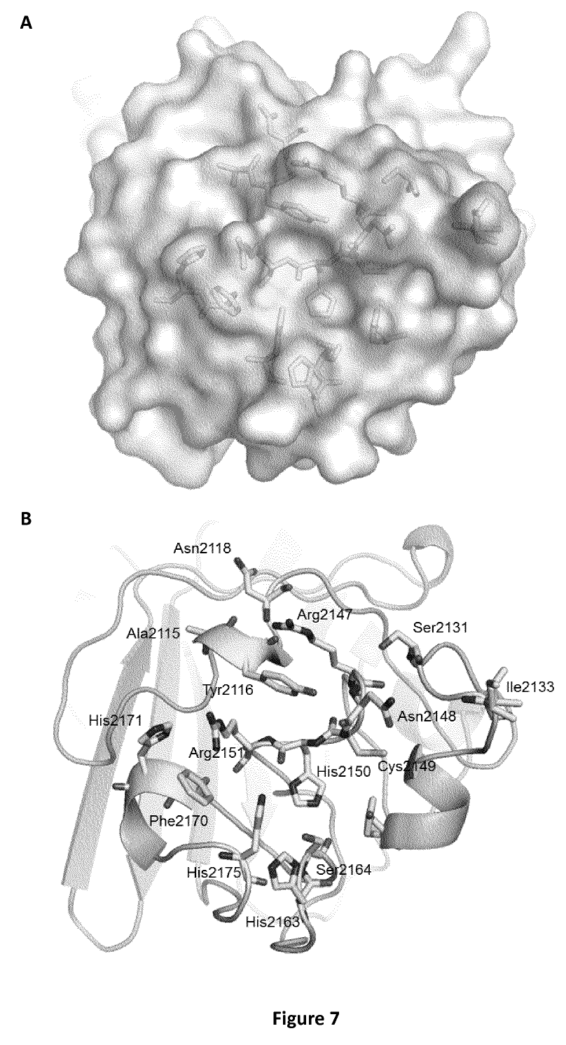

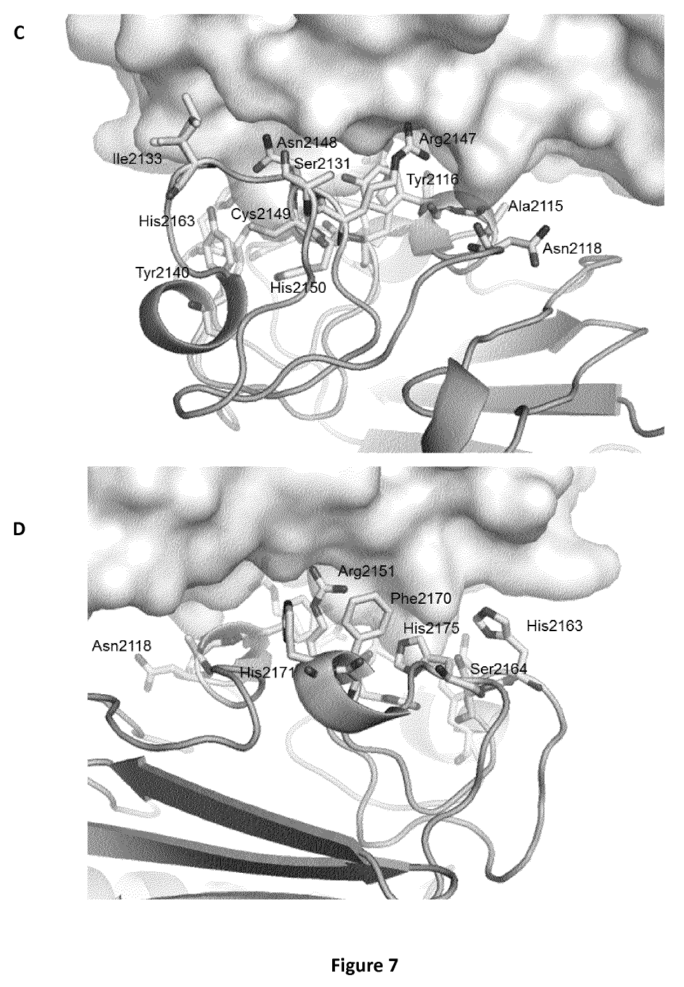

The positions of these residues and their interaction with the Fab′ are shown in FIG. 7. These residues define the epitope of the C3 antibody.

### Modelling Structure

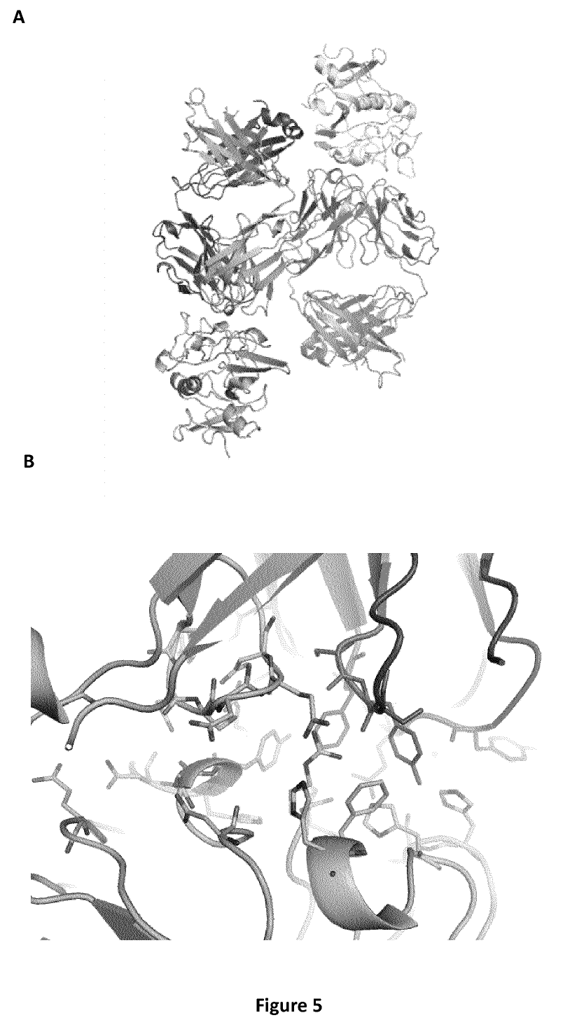

The crystal of the Fab′-Tenascin C complex was then used for structure modelling. The structure itself was determined by molecular replacement. FIG. 5 shows the modelled structure of the Fab′-Tenascin complex. As can be seen, 2 complexes were observed in an asymmetric unit.

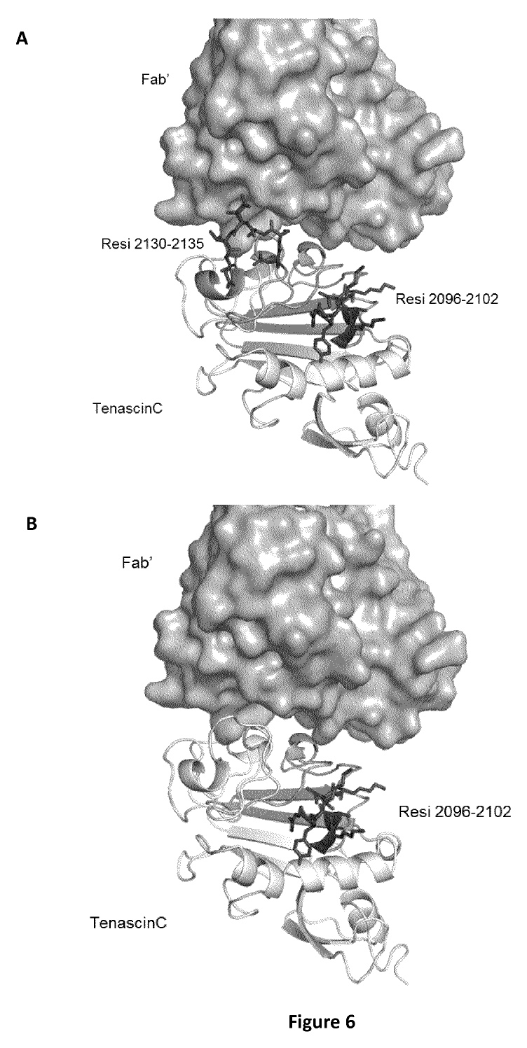

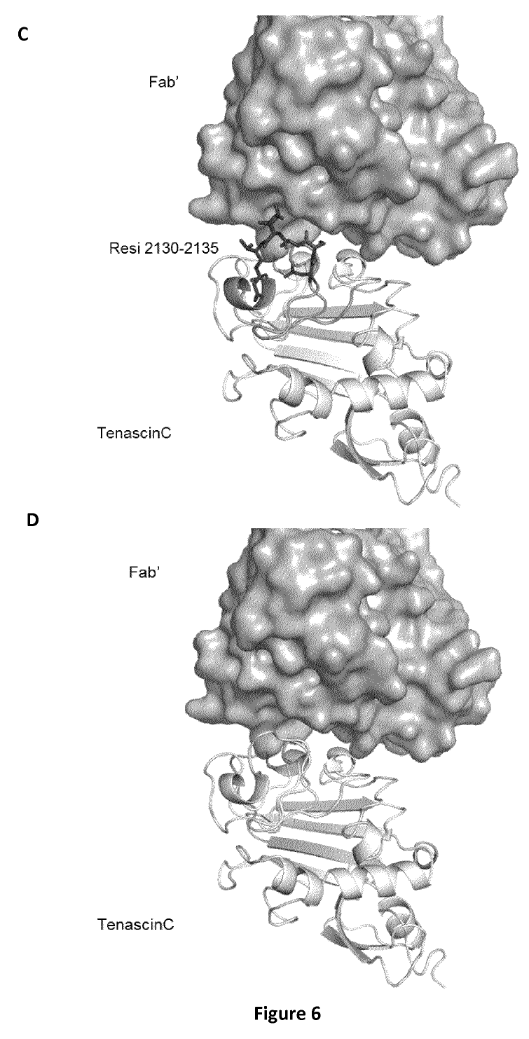

FIG. 6 shows the various residues of Tenascin-C that are of interest. FIG. 6A is an overview of FIGS. 6B to 6D. Residues 2096-2102 and 2130-2135 are highlighted in black by stick representation. Residues 2071-2148 and 2170-2193 are highlighted in dark grey in cartoon representation. Residues 2194-2201 are not visible within the structure and are shown in light grey. The Fab′ is shown by surface representation. The residues are defined using the sequence given in Uniprot ID P24821.

These residues are also shown in the amino acid sequence below from SEQ ID NO: 1 with residues 2096-2102 and 2130-2135 in bold and residues 2194-2201 in italics.

ELRVDLRDHGETAFAVYDKFSVGDAKTRYKLKVEGYSGTAGDSMAYHNGR
SFST (residues 2071-2124)
RSFSTFDKDTDSAITNCALSYKGAFWYRN (residues 2120-2148)
FHWKGHEHSIQFAEMKLRPSNFRNLEGRRKRA (residues 2170-
2201)

Similarly, a number of residues within the Fab′ which are located within 4 Å of the tenascin C construct were identified:

| Light | Heavy |
|---|---|
| chain | chain |
| Tyr28 | Tyr100 |
| Gln30 | Gln101 |
| Gly31 | Glu104 |
| Phe32 | Asp105 |
| Asp50 | |
| Ala51 | |
| Ser52 | |
| Asn53 | |
| Gly64 | |
| Ser91 | |
| Tyr92 | |

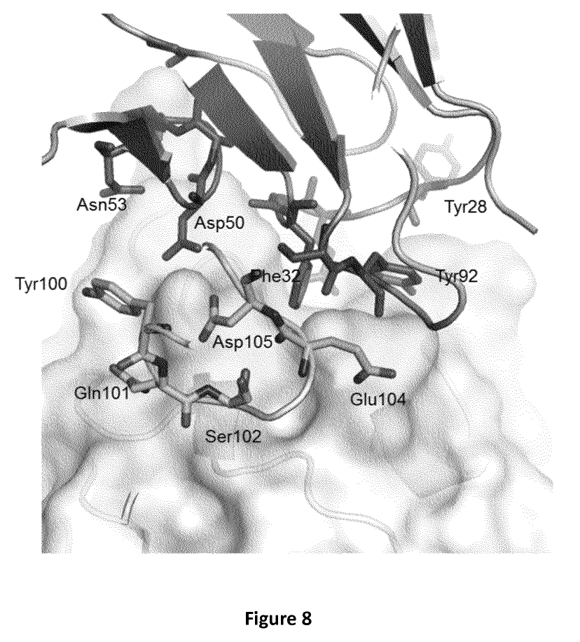

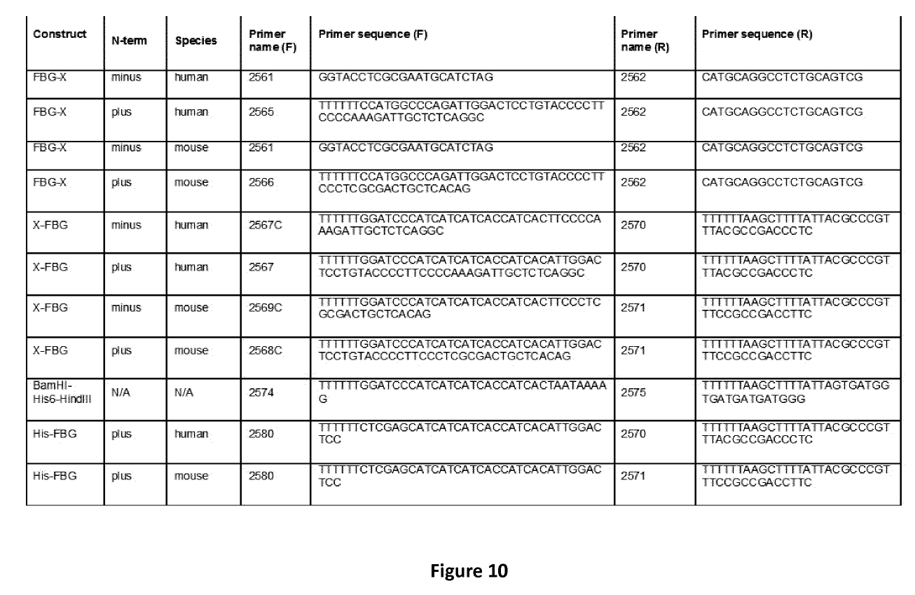

The positions of these residues on the Fab′-tenascin complex structure are shown in FIG. 8.

1.-28. (canceled)

29. A neutralising conformational epitope of a Tenascin-C which comprises at least 6 amino acids selected from group Ala2115, Tyr2116, Asn2118, Ser2131, Ile2133, Tyr2140, Arg2147, Asn2148, Cys2149, His2150, Arg2151, His2163, Ser2164, Phe2170, His2171, and His2175, wherein said amino acid residue positions are those of SEQ ID NO: 1,
wherein said epitope comprises at least Arg2147, Arg2151 and Ile2133, and
residues in the epitope are within 4 Å of the C3 antibody binding domain shown in SEQ ID NO: 2 and 3, and
inhibition or blocking the epitope neutralises the biological signalling activity of the FBG domain of Tenascin C.

30. A neutralising epitope of Tenascin-C according to claim 29, which comprises the linear sequence of amino acids Arg2147, Asn2148, Cys2149, His2150, and Arg2151.

31. A neutralising epitope of Tenascin-C according to claim 29, which comprises polar residue His2163.

32. A neutralising epitope of Tenascin-C according to claim 29, which comprises Cys2149.

33. A neutralising epitope of Tenascin-C according to claim 29, which comprises hydrophobic residue His2171.

34. A neutralising epitope of Tenascin-C according to claim 29, which comprises the polar amino acid Tyr2116.

35. A neutralising epitope of Tenascin-C according to claim 29, which comprises the hydrophobic residue Phe2170.

36. A neutralising epitope of Tenascin-C according to claim 29, which comprises Ile2133, Arg2147, Asn2148, His2163, and Phe2170.

37. A neutralising epitope of Tenascin-C according to claim 36, which further comprises amino acid residue Ser2131.

38. A neutralising epitope of Tenascin-C according to claim 36, which further comprises amino acid residue Tyr2116.

39. A neutralising epitope of Tenascin-C according to claim 29 comprising the residues Ala2115, Asn2118, Ser2131, Arg2147, Asn2148, Cys2149, His2150, Arg2151, Ser2164, His2171, and His2175.

40. A neutralising epitope of Tenascin-C according to claim 29 comprising the residues Ala2115, Tyr2116, Asn2118, Ser2131, Tyr2140, Arg2147, Asn2148, Cys2149, His2150, Arg2151, His2163, Ser2164, Phe2170, His2171 and His2175.

41. A neutralising epitope of Tenascin-C according to claim 29 comprising the residues Ala2115, Tyr2116, Asn2118, Ser2131, Ile2133, Tyr2140, Arg2147, Asn2148, Cys2149, His2150, Arg2151, His2163, Ser2164, Phe2170, His2171, His2175.

42. A neutralising epitope of Tenascin-C according to claim 29, which comprises 7, 8, 9, 10, 11, 12, 13, 14 15 or 16 of said amino acid residues.

43. A neutralising antibody or binding fragment thereof, which binds an epitope defined in claim 29, wherein the antibody does not comprise the CDRs of antibody C3, B12 or 2A5.

44. A neutralising antibody or binding fragment according to claim 43, wherein at least amino acid 101, according to the Kabat numbering system, of the heavy chain is a polar amino acid, for example Gln.

45. A neutralising antibody or binding fragment according to claim 43, wherein at least amino acid 102, according to the Kabat numbering system, of the heavy chain is polar, for example serine.

46. A neutralising antibody or binding fragment, according to claim 43, wherein at least amino acid 104, according to the Kabat numbering system, of the heavy chain is charged, for example Glu.

47. A neutralising antibody or binding fragment, according to claim 43, wherein at least amino acid 105, according to the Kabat numbering system, is a negatively charged amino acid, for example Asp.

48. A neutralising antibody or binding fragment, according to claim 43, wherein the antibody or binding fragment is human or humanised.

49. A neutralising antibody or binding fragment according to claim 43, wherein the affinity is in the range 5 pM to 500 nM as measured by surface plasmon resonance.

50. A pharmaceutical composition comprising an antibody or binding fragment according to claim 43 and a diluent, excipient and/or carrier.

51. A method of treatment, comprising administering to a subject, a pharmaceutical composition according to claim 50.

52. A method of treatment, comprising administering to a subject having chronic inflammation a pharmaceutical composition according to claim 50.


SEQUENCE LISTING (148 sequences)

SEQ ID NO: 1 - protein, 2201 aa, Artificial Sequence
  Tenascin C amino acid sequence
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGVDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDITGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 2 - protein, 446 aa, Artificial Sequence
  Fab′ heavy chain sequence
QVQLVESGGGLVQPGRSLRLSCAASGFTFDDYAMHWVRQAPGKGLEWVSGISGSGGSTYYADSVKGRFTISRDNAKNSLYLQMNSLRAEDTALYYCAKSYQSDEDAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 214 aa, Artificial Sequence
  Fab′ light chain sequence
DIQMTQSPSSLSASVGDRVTITCRASQYIQGFLNWYQQKPGKAPKLLIYDASNLETGVPSRFSGSGSGTDFTLTISSLQPEDFATYYCQQSYSTPQTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 5 aa, Artificial Sequence
  Fab′ VH CDR1
DYAMH

SEQ ID NO: 5 - protein, 17 aa, Artificial Sequence
  Fab′ VH CDR2
GISGSGGSTYYADSVKG

SEQ ID NO: 6 - protein, 11 aa, Artificial Sequence
  Fab′ VH CDR3
SYQSDEDAFDI

SEQ ID NO: 7 - protein, 11 aa, Artificial Sequence
  Fab′ VL CDR1
RASQYIQGFLN

SEQ ID NO: 8 - protein, 7 aa, Artificial Sequence
  Fab′ VL CDR2
DASNLET

SEQ ID NO: 9 - protein, 9 aa, Artificial Sequence
  Fab′ VL CDR3
QQSYSTPQT

SEQ ID NO: 10 - protein, 238 aa, Artificial Sequence
  Tenascin C construct
QILEHHHHHHIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 11 - protein, 5 aa, Artificial Sequence
  VH CDR1
ELSMH

SEQ ID NO: 12 - protein, 17 aa, Artificial Sequence
  VH CDR2
GFDPEDGETIYAQKFQG

SEQ ID NO: 13 - protein, 10 aa, Artificial Sequence
  VH CDR3
AQKETYALTY

SEQ ID NO: 14 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAQKETYALTYWGQGTLVTVSS

SEQ ID NO: 15 - protein, 11 aa, Artificial Sequence
  VH CDR3
DKYPVLDTFDI

SEQ ID NO: 16 - protein, 7 aa, Artificial Sequence
  VL CDR2
AASTLQD

SEQ ID NO: 17 - protein, 9 aa, Artificial Sequence
  VL CDR3
QQSYSTPWT

SEQ ID NO: 18 - protein, 108 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCQQSYSTPWTFGQGTKVEIKR

SEQ ID NO: 19 - protein, 107 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCQQSYSTPWTFGQGTKVEIK

SEQ ID NO: 20 - protein, 120 aa, Artificial Sequence
  VH amino acid sequence
QVQLVESGGGLVQPGRSLRLSCAASGFTFDDYAMHWVRQAPGKGLEWVSGISGSGGSTYYADSVKGRFTISRDNAKNSLYLQMNSLRAEDTALYYCAKDKYPVLDTFDIWGQGTMVTVSS

SEQ ID NO: 21 - protein, 11 aa, Artificial Sequence
  VH CDR3
ALARGHDTFDI

SEQ ID NO: 22 - protein, 11 aa, Artificial Sequence
  VH CDR3
DISAVPDTFDI

SEQ ID NO: 23 - protein, 120 aa, Artificial Sequence
  VH amino acid sequence
QVQLVESGGGLVQPGRSLRLSCAASGFTFDDYAMHWVRQAPGKGLEWVSGISGSGGSTYYADSVKGRFTISRDNAKNSLYLQMNSLRAEDTALYYCAKDISAVPDTFDIWGQGTMVTVSS

SEQ ID NO: 24 - protein, 120 aa, Artificial Sequence
  VH amino acid sequence
QVQLVESGGGLVQPGRSLRLSCAASGFTFDDYAMHWVRQAPGKGLEWVSGISGSGGSTYYADSVKGRFTISRDNAKNSLYLQMNSLRAEDTALYYCAKALARGHDTFDIWGQGTMVTVSS

SEQ ID NO: 25 - protein, 108 aa, Artificial Sequence
  VL amino acid sequence
DIQMTQSPASLPTPVGDRVTITCRASQYIQGFLNWYQQKPGKAPKLLIYDASNLETGVPSRFSGSGSGTDFTLTISSLQPEDFATYYCQQSYSTPQTFGQGTKVDIKR

SEQ ID NO: 26 - protein, 107 aa, Artificial Sequence
  VL amino acid sequence
DIQMTQSPASLPTPVGDRVTITCRASQYIQGFLNWYQQKPGKAPKLLIYDASNLETGVPSRFSGSGSGTDFTLTISSLQPEDFATYYCQQSYSTPQTFGQGTKVDIK

SEQ ID NO: 27 - protein, 5 aa, Artificial Sequence
  VH CDR1
SYGIS

SEQ ID NO: 28 - protein, 17 aa, Artificial Sequence
  VH CDR2
WISAYNGNTNYAQKLQG

SEQ ID NO: 29 - protein, 8 aa, Artificial Sequence
  VH CDR3
NQDSSSDY

SEQ ID NO: 30 - protein, 117 aa, Artificial Sequence
  VH amino acid sequence
EVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARNQDSSSDYWGQGTLVTVSS

SEQ ID NO: 31 - protein, 9 aa, Artificial Sequence
  VL CDR3
QQSYSTLQT

SEQ ID NO: 32 - protein, 108 aa, Artificial Sequence
  VL amino acid sequence
DIQMTQSPASLPTSVGDRVTITCRASQYIQGFLNWYQQKPGKAPKLLIYDASNLETGVPSRFSGSGSGTDFTLTISSLQPEDFATYYCQQSYSTLQTFGQGTKVDIKR

SEQ ID NO: 33 - protein, 107 aa, Artificial Sequence
  VL amino acid sequence
DIQMTQSPASLPTSVGDRVTITCRASQYIQGFLNWYQQKPGKAPKLLIYDASNLETGVPSRFSGSGSGTDFTLTISSLQPEDFATYYCQQSYSTLQTFGQGTKVDIK

SEQ ID NO: 34 - protein, 5 aa, Artificial Sequence
  VH CDR1
SYGMH

SEQ ID NO: 35 - protein, 17 aa, Artificial Sequence
  VH CDR2
VISYDGSNKYYADSVKG

SEQ ID NO: 36 - protein, 16 aa, Artificial Sequence
  VH CDR3
EGYDQLFSAESNAFDI

SEQ ID NO: 37 - protein, 125 aa, Artificial Sequence
  VH amino acid sequence
EVQLLESGGGLVQPGGSLRLSCAASGFTFSSYGMHWVRQAPGKGLEWVAVISYDGSNKYYADSVKGRFTISRGNSKNTLYLQMNSLKAEDTAVYYCAREGYDQLFSAESNAFDIWGQGTLVTVSS

SEQ ID NO: 38 - protein, 13 aa, Artificial Sequence
  VL CDR1
TRSSGSIASYFVQ

SEQ ID NO: 39 - protein, 7 aa, Artificial Sequence
  VL CDR2
EDNQRPS

SEQ ID NO: 40 - protein, 9 aa, Artificial Sequence
  VL CDR3
QSYDSSNWV

SEQ ID NO: 41 - protein, 113 aa, Artificial Sequence
  VL amino acid sequence
NFMLAQPHSVSESPGKTVTISCTRSSGSIASYFVQWFQQRPGSAPTAVIYEDNQRPSGVPDRFSGSIDSSSNSASLTISGLKTEDEADYYCQSYDSSNWVFGGGTKVTVLGQP

SEQ ID NO: 42 - protein, 110 aa, Artificial Sequence
  VL amino acid sequence
NFMLAQPHSVSESPGKTVTISCTRSSGSIASYFVQWFQQRPGSAPTAVIYEDNQRPSGVPDRFSGSIDSSSNSASLTISGLKTEDEADYYCQSYDSSNWVFGGGTKVTVL

SEQ ID NO: 43 - protein, 11 aa, Artificial Sequence
  VH CDR3
VMSSMEDAFDI

SEQ ID NO: 44 - protein, 120 aa, Artificial Sequence
  VH amino acid sequence
QVQLVESGGGLVQPGRSLRLSCAASGFTFDDYAMHWVRQAPGKGLEWVSGISGSGGSTYYADSVKGRFTISRDNAKNSLYLQMNSLRAEDTALYYCAKVMSSMEDAFDIWGQGTMVTVSS

SEQ ID NO: 45 - protein, 11 aa, Artificial Sequence
  VH CDR3
DISAVMDVPQT

SEQ ID NO: 46 - protein, 11 aa, Artificial Sequence
  VH CDR3
GQKGEGDTFDI

SEQ ID NO: 47 - protein, 120 aa, Artificial Sequence
  VH amino acid sequence
QVQLVESGGGLVQPGRSLRLSCAASGFTFDDYAMHWVRQAPGKGLEWVSGISGSGGSTYYADSVKGRFTISRDNAKNSLYLQMNSLRAEDTALYYCAKGQKGEGDTFDIWGQGTMVTVSS

SEQ ID NO: 48 - protein, 120 aa, Artificial Sequence
  VH amino acid sequence
QVQLVESGGGLVQPGRSLRLSCAASGFTFDDYAMHWVRQAPGKGLEWVSGISGSGGSTYYADSVKGRFTISRDNAKNSLYLQMNSLRAEDTALYYCAKDISAVMDVPQTWGQGTMVTVSS

SEQ ID NO: 49 - protein, 11 aa, Artificial Sequence
  VH CDR3
GTRGEGDTFDI

SEQ ID NO: 50 - protein, 120 aa, Artificial Sequence
  VH amino acid sequence
QVQLVESGGGLVQPGRSLRLSCAASGFTFDDYAMHWVRQAPGKGLEWVSGISGSGGSTYYADSVKGRFTISRDNAKNSLYLQMNSLRAEDTALYYCAKGTRGEGDTFDIWGQGTMVTVSS

SEQ ID NO: 51 - protein, 11 aa, Artificial Sequence
  VH CDR3
VMRTGLDTFDI

SEQ ID NO: 52 - protein, 120 aa, Artificial Sequence
  VH amino acid sequence
QVQLVESGGGLVQPGRSLRLSCAASGFTFDDYAMHWVRQAPGKGLEWVSGISGSGGSTYYADSVKGRFTISRDNAKNSLYLQMNSLRAEDTALYYCAKSYQSDEDAFDIWGQGTMVTVSS

SEQ ID NO: 53 - protein, 11 aa, Artificial Sequence
  VH CDR3
GTVGEGDTFDI

SEQ ID NO: 54 - protein, 120 aa, Artificial Sequence
  VH amino acid sequence
QVQLVESGGGLVQPGRSLRLSCAASGFTFDDYAMHWVRQAPGKGLEWVSGISGSGGSTYYADSVKGRFTISRDNAKNSLYLQMNSLRAEDTALYYCAKGTVGEGDTFDIWGQGTMVTVSS

SEQ ID NO: 55 - protein, 120 aa, Artificial Sequence
  VH amino acid sequence
QVQLVESGGGLVQPGRSLRLSCAASGFTFDDYAMHWVRQAPGKGLEWVSGISGSGGSTYYADSVKGRFTISRDNAKNSLYLQMNSLRAEDTALYYCAKVMRTGLDTFDIWGQGTMVTVSS

SEQ ID NO: 56 - protein, 10 aa, Artificial Sequence
  VH CDR3
QRYVWEALTY

SEQ ID NO: 57 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATQRYVWEALTYWGQGTLVTVSS

SEQ ID NO: 58 - protein, 10 aa, Artificial Sequence
  VH CDR3
AQADPHLFTY

SEQ ID NO: 59 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAQADPHLFTYWGQGTLVTVSS

SEQ ID NO: 60 - protein, 10 aa, Artificial Sequence
  VH CDR3
GRFVWEALTY

SEQ ID NO: 61 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATGRFVWEALTYWGQGTLVTVSS

SEQ ID NO: 62 - protein, 10 aa, Artificial Sequence
  VH CDR3
AQKETLGNAI

SEQ ID NO: 63 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAQKETLGNAIWGQGTLVTVSS

SEQ ID NO: 64 - protein, 10 aa, Artificial Sequence
  VH CDR3
AQSPWSGMTY

SEQ ID NO: 65 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAQSPWSGMTYWGQGTLVTVSS

SEQ ID NO: 66 - protein, 10 aa, Artificial Sequence
  VH CDR3
YTLDNMALTY

SEQ ID NO: 67 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATYTLDNMALTYWGQGTLVTVSS

SEQ ID NO: 68 - protein, 10 aa, Artificial Sequence
  VH CDR3
AQKENIANRH

SEQ ID NO: 69 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAQKENIANRHWGQGTLVTVSS

SEQ ID NO: 70 - protein, 10 aa, Artificial Sequence
  VH CDR3
AQPTALANTY

SEQ ID NO: 71 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAQPTALANTYWGQGTLVTVSS

SEQ ID NO: 72 - protein, 10 aa, Artificial Sequence
  VH CDR3
AQLPYLAQTY

SEQ ID NO: 73 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAQLPYLAQTYWGQGTLVTVSS

SEQ ID NO: 74 - protein, 10 aa, Artificial Sequence
  VH CDR3
AQPVWAPGTY

SEQ ID NO: 75 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAQPVWAPGTYWGQGTLVTVSS

SEQ ID NO: 76 - protein, 10 aa, Artificial Sequence
  VH CDR3
AQKEWLPDVT

SEQ ID NO: 77 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAQKEWLPDVTWGQGTLVTVSS

SEQ ID NO: 78 - protein, 10 aa, Artificial Sequence
  VH CDR3
AQIHPLGLTY

SEQ ID NO: 79 - protein, 119 aa, Artificial Sequence
  VH amino acid sequence
EVRLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAQIHPLGLTYWGQGTLVTVSS

SEQ ID NO: 80 - protein, 9 aa, Artificial Sequence
  VL CDR3
QNQYAGPWT

SEQ ID NO: 81 - protein, 108 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCQNQYAGPWTFGQGTKVEIKR

SEQ ID NO: 82 - protein, 107 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCQNQYAGPWTFGQGTKVEIK

SEQ ID NO: 83 - protein, 9 aa, Artificial Sequence
  VL CDR3
QNQYTGPWT

SEQ ID NO: 84 - protein, 108 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCQNQYTGPWTFGQGTKVEIKR

SEQ ID NO: 85 - protein, 107 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCQNQYTGPWTFGQGTKVEIK

SEQ ID NO: 86 - protein, 9 aa, Artificial Sequence
  VL CDR3
QNQYRGPWT

SEQ ID NO: 87 - protein, 108 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCQNQYRGPWTFGQGTKVEIKR

SEQ ID NO: 88 - protein, 107 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCQNQYRGPWTFGQGTKVEIK

SEQ ID NO: 89 - protein, 9 aa, Artificial Sequence
  VL CDR3
LHHYRAPWT

SEQ ID NO: 90 - protein, 108 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCLHHYRAPWTFGQGTKVEIKR

SEQ ID NO: 91 - protein, 107 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCLHHYRAPWTFGQGTKVEIK

SEQ ID NO: 92 - protein, 9 aa, Artificial Sequence
  VL CDR3
MHHYRAPWT

SEQ ID NO: 93 - protein, 108 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCMHHYRAPWTFGQGTKVEIKR

SEQ ID NO: 94 - protein, 107 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCMHHYRAPWTFGQGTKVEIK

SEQ ID NO: 95 - protein, 9 aa, Artificial Sequence
  VL CDR3
MHHYRSPWT

SEQ ID NO: 96 - protein, 108 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCMHHYRSPWTFGQGTKVEIKR

SEQ ID NO: 97 - protein, 107 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCMHHYRSPWTFGQGTKVEIK

SEQ ID NO: 98 - protein, 9 aa, Artificial Sequence
  VL CDR3
MQHYDGPWT

SEQ ID NO: 99 - protein, 108 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCMQHYDGPWTFGQGTKVEIKR

SEQ ID NO: 100 - protein, 107 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCMQHYDGPWTFGQGTKVEIK

SEQ ID NO: 101 - protein, 10 aa, Artificial Sequence
  VL CDR3
LHHYRSPTWT

SEQ ID NO: 102 - protein, 9 aa, Artificial Sequence
  VL CDR3
LHHYRSPWT

SEQ ID NO: 103 - protein, 109 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCLHHYRSPTWTFGQGTKVEIKR

SEQ ID NO: 104 - protein, 107 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCLHHYRSPWTFGQGTKVEIK

SEQ ID NO: 105 - protein, 9 aa, Artificial Sequence
  VL CDR3
LHHYREPWT

SEQ ID NO: 106 - protein, 108 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCLHHYREPWTFGQGTKVEIKR

SEQ ID NO: 107 - protein, 107 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCLHHYREPWTFGQGTKVEIK

SEQ ID NO: 108 - protein, 9 aa, Artificial Sequence
  VL CDR3
LHHYKSPWT

SEQ ID NO: 109 - protein, 108 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCLHHYKSPWTFGQGTKVEIKR

SEQ ID NO: 110 - protein, 107 aa, Artificial Sequence
  VL amino acid sequence
DIQMTQSPASLPTSVGDRVTITCRASQYIQGFLNWYQQKPGKAPRLLIYAASTLQDGVPSRFSGSGYGTDFTLTISSLQPEDFATYYCLHHYKSPWTFGQGTKVEIK

SEQ ID NO: 111 - protein, 446 aa, Artificial Sequence
  IgG4 165_13_C3
QVQLVESGGGLVQPGRSLRLSCAASGFTFDDYAMHWVRQAPGKGLEWVSGISGSGGSTYYADSVKGRFTISRDNAKNSLYLQMNSLRAEDTALYYCAKSYQSDEDAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFPSDIAVEWESNGQPENNYKTTPPVLDSDGSFFLYSRLTVDKSRWQEGNVFSCSVMHEALHNHYTQKSLSLSLGK

SEQ ID NO: 112 - protein, 228 aa, Artificial Sequence
  Amino acid sequence of human tenascin-C FBG domain
IGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 113 - protein, 228 aa, Artificial Sequence
  Amino acid sequence of mouse tenascin-C FBG domain
IGLLYPFPRDCSQAMLNGDTTSGLYTIYINGDKTQALEVYCDMTSDGGGWIVFLRRKNGREDFYRNWKAYAAGFGDRREEFWLGLDNLSKITAQGQYELRVDLQDHGESAYAVYDRFSVGDAKSRYKLKVEGYSGTAGDSMNYHNGRSFSTYDKDTDSAITNCALSYKGAFWYKNCHRVNLMGRYGDNNHSQGVNWFHWKGHEYSIQFAEMKLRPSNFRNLEGRRKRA

SEQ ID NO: 114 - protein, 228 aa, Artificial Sequence
  Amino acid sequence of rat tenascin-C FBG domain
IGLLYPFPRDCSQAMLNGDTTSGLYTIYINGDKTQALEVYCDMTSDGGGWIVFLRRKNGREDFYRNWKAYATGFGDRREEFWLGLDNLSKITAQGQYELRVDLQDHGESAYAVYDRFSVGDAKSRYKLKVEGYSGTAGDSMNYHNGRSFSTYDKDTDSAITNCALSYKGAFWYKNCHRVNLMGRYGDNNHSQGVNWFHWKGHEYSIQFAEMKLRPSNFRNLEGRRKRA

SEQ ID NO: 115 - protein, 228 aa, Artificial Sequence
  Amino acid sequence of dog tenascin-C FBG domain
IGLLYPFPRDCSQAMLNGDTTSGLYTIYLNGDKAQALEVYCDMTSDGGGWIVFLRRKNGREDFYRNWKAYAAGFGDRREEFWLGLDNLHKITAQGQYELRVDLRDHGKTAYAVYDRFSVGDAKTRYKLKVEGYSGTAGDSMAYHNGRSFSTFDKDTDSAITNCALSYKGAFWYKNCHRVNLMGRYGDNNHSQGVNWFHWKGHEYSIQFAEMKLRPSNFRNLEGRRKRA

SEQ ID NO: 116 - protein, 227 aa, Artificial Sequence
  Amino acid sequence of human tenascin-R FBG domain
FPHPQDCAQHLMNGDTLSGVYPIFLNGELSQKLQVYCDMTTDGGGWIVFQRRQNGQTDFFRKWADYRVGFGNVEDEFWLGLDNIHRITSQGRYELRVDMRDGQEAAFASYDRFSVEDSRNLYKLRIGSYNGTAGDSLSYHQGRPFSTEDRDNDVAVTNCAMSYKGAWWYKNCHRTNLNGKYGESRHSQGINWYHWKGHEFSIPFVEMKMRPYNHRLMAGRKRQSLQF

SEQ ID NO: 117 - protein, 119 aa, Artificial Sequence
  Framework Germlined VH amino acid sequence
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAQKETYALTYWGQGTLVTVSS

SEQ ID NO: 118 - protein, 108 aa, Artificial Sequence
  Framework Germlined VL amino acid sequence
DIQMTQSPSSLSASVGDRVTITCRASQYIQGFLNWYQQKPGKAPKLLIYAASSLQSGVPSRFSGSGSGTDFTLTISSLQPEDFATYYCQQSYSTPWTFGQGTKVEIKR

SEQ ID NO: 119 - protein, 107 aa, Artificial Sequence
  Framework Germlined VL amino acid sequence
DIQMTQSPSSLSASVGDRVTITCRASQYIQGFLNWYQQKPGKAPKLLIYAASSLQSGVPSRFSGSGSGTDFTLTISSLQPEDFATYYCQQSYSTPWTFGQGTKVEIK

SEQ ID NO: 120 - protein, 7 aa, Artificial Sequence
  VL CDR2
AASSLQS

SEQ ID NO: 121 - protein, 120 aa, Artificial Sequence
  Framework Germlined VH amino acid sequence
EVQLVESGGGLVQPGRSLRLSCAASGFTFDDYAMHWVRQAPGKGLEWVSGISGSGGSTYYADSVKYRFTISRDNAKNSLYLQMNSLRAEDTALYYCAKDISAVPDTFDIWGQGTMVTVSS

SEQ ID NO: 122 - protein, 17 aa, Artificial Sequence
  VH CDR2
GISGSGGSTYYADSVKY

SEQ ID NO: 123 - protein, 108 aa, Artificial Sequence
  Framework Germlined VL amino acid sequence
DIQMTQSPSSLSASVGDRVTITCRASQYIQGFLNWYQQKPGKAPKLLIYDASSLQSGVPSRFSGSGSGTDFTLTISSLQPEDFATYYCQQSYSTPQTFGQGTKVDIKR

SEQ ID NO: 124 - protein, 107 aa, Artificial Sequence
  Framework Germlined VL amino acid sequence
DIQMTQSPSSLSASVGDRVTITCRASQYIQGFLNWYQQKPGKAPKLLIYDASSLQSGVPSRFSGSGSGTDFTLTISSLQPEDFATYYCQQSYSTPQTFGQGTKVDIK

SEQ ID NO: 125 - protein, 7 aa, Artificial Sequence
  VL CDR2
DASSLQS

SEQ ID NO: 126 - protein, 117 aa, Artificial Sequence
  Framework Germlined VH amino acid sequence
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARNQDSSSDYWGQGTLVTVSS

SEQ ID NO: 127 - protein, 108 aa, Artificial Sequence
  Framework Germlined VL amino acid sequence
DIQMTQSPSSLSASVGDRVTITCRASQYIQGFLNWYQQKPGKAPKLLIYDASSLQSGVPSRFSGSGSGTDFTLTISSLQPEDFATYYCQQSYSTLQTFGQGTKVEIKR

SEQ ID NO: 128 - protein, 107 aa, Artificial Sequence
  Framework Germlined VL amino acid sequence
DIQMTQSPSSLSASVGDRVTITCRASQYIQGFLNWYQQKPGKAPKLLIYDASSLQSGVPSRFSGSGSGTDFTLTISSLQPEDFATYYCQQSYSTLQTFGQGTKVEIK

SEQ ID NO: 129 - protein, 125 aa, Artificial Sequence
  Framework germlined VH amino acid sequence
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAREGYDQLFSAESNAFDIWGQGTLVTVSS

SEQ ID NO: 130 - protein, 113 aa, Artificial Sequence
  Framework germlined VL amino acid sequence
NFMLTQPHSVSESPGKTVTISCTRSSGSIASYFVQWYQQRPGSSPTTVIYEDNQRPSGVPDRFSGSIDSSSNSASLTISGLKTEDEADYYCQSYDSSNWVFGGGTKLTVLGQP

SEQ ID NO: 131 - protein, 110 aa, Artificial Sequence
  Framework germlined VL amino acid sequence
NFMLTQPHSVSESPGKTVTISCTRSSGSIASYFVQWYQQRPGSSPTTVIYEDNQRPSGVPDRFSGSIDSSSNSASLTISGLKTEDEADYYCQSYDSSNWVFGGGTKLTVL

SEQ ID NO: 132 - protein, 54 aa, Artificial Sequence
  TENASCIN FRAGMENT (residues 2071-2124)
ELRVDLRDHGETAFAVYDKFSVGDAKTRYKLKVEGYSGTAGDSMAYHNGRSFST

SEQ ID NO: 133 - protein, 29 aa, Artificial Sequence
  TENASCIN FRAGMENT (residues 2120-2148)
RSFSTFDKDTDSAITNCALSYKGAFWYRN

SEQ ID NO: 134 - protein, 32 aa, Artificial Sequence
  TENASCIN FRAGMENT (residues 2170-2201)
FHWKGHEHSIQFAEMKLRPSNFRNLEGRRKRA

SEQ ID NO: 135 - DNA, 22 bp, Artificial Sequence
  PRIMER
ggtacctcgcgaatgcatctag

SEQ ID NO: 136 - DNA, 58 bp, Artificial Sequence
  PRIMER
ttttttccatggcccagattggactcctgtaccccttccccaaagattgctctcaggc

SEQ ID NO: 137 - DNA, 55 bp, Artificial Sequence
  PRIMER
ttttttcctggcccagattggactcctgtaccccttccctcgcgactgctcacag

SEQ ID NO: 138 - DNA, 20 bp, Artificial Sequence
  PRIMER
catgcaggcctctgcagtcg

SEQ ID NO: 139 - DNA, 39 bp, Artificial Sequence
  PRIMER
ttttttaagcttttattacgcccgtttacgccgaccctc

SEQ ID NO: 140 - DNA, 39 bp, Artificial Sequence
  PRIMER
ttttttaagcttttattacgcccgtttccgccgaccttc

SEQ ID NO: 141 - DNA, 38 bp, Artificial Sequence
  PRIMER
ttttttaagcttttattagtgatggtgatgatgatggg

SEQ ID NO: 142 - DNA, 56 bp, Artificial Sequence
  PRIMER
ttttttccatggcccagattggactcctgtaccccttccctcgcgactgctcacag

SEQ ID NO: 143 - DNA, 53 bp, Artificial Sequence
  PRIMER
ttttttggatcccatcatcatcaccatcacttccccaaagattgctctcaggc

SEQ ID NO: 144 - DNA, 71 bp, Artificial Sequence
  PRIMER
ttttttggatcccatcatcatcaccatcacattggactcctgtaccccttccccaaagattgctctcaggc

SEQ ID NO: 145 - DNA, 51 bp, Artificial Sequence
  PRIMER
ttttttggatcccatcatcatcaccatcacttccctcgcgactgctcacag

SEQ ID NO: 146 - DNA, 69 bp, Artificial Sequence
  PRIMER
ttttttggatcccatcatcatcaccatcacattggactcctgtaccccttccctcgcgactgctcacag

SEQ ID NO: 147 - DNA, 39 bp, Artificial Sequence
  PRIMER
ttttttggatcccatcatcatcaccatcactaataaaag

SEQ ID NO: 148 - DNA, 36 bp, Artificial Sequence
  PRIMER
ttttttccgagcatcatcatcaccatcacattggac